(19) United States
(12) Patent Application Publication
(10) Pub. No.: US 2024/0307488 A1
(43) Pub. Date: Sep. 19, 2024

(54) ANNEXIN TO INHIBIT BACTERIA

(71) Applicant: University of Virginia Patent Foundation, Charlottesville, VA (US)

(72) Inventor: Carl E. Creutz, Earlysville, VA (US)

(21) Appl. No.: 18/604,185

(22) Filed: Mar. 13, 2024

Related U.S. Application Data
(60) Provisional application No. 63/489,841, filed on Mar. 13, 2023.

(51) Int. Cl. A61K 38/17; A61K 45/06; C12N 15/63

(52) U.S. Cl. CPC A61K 38/1738; A61K 45/06; C12N 15/63

(57) ABSTRACT

Provided herein are compositions comprising an annexin polypeptide or subunit thereof and methods using said composition.

### PRIORITY

This application claims the benefit of U.S. Provisional Application No. 63/489,841, filed Mar. 13, 2023, the content of which is herein incorporated by reference in its entirety.

### BACKGROUND

Treatment of bacteria with beta-lactam antibiotics or other environmental stressors can impair the process of cytokinesis, the final step in cell division, leading to the formation of a filamentous form of the bacteria that can evade the immune system.

### SUMMARY

The expression of a mammalian calcium-dependent, membrane-binding protein, bovine annexin A4, in bacteria, such as E. coli, was found to reverse the inhibitory effects on cytokinesis of the antibiotics, such as beta-lactam ampicillin, piperacillin, and cephalexin. This novel activity of the annexin was blocked by mutation of calcium binding sites in the annexin, indicating roles for calcium binding to the annexin and the binding of the annexin to membranes in restoring cytokinesis. The filamentous/blocked form of the bacteria has been reported to be more resistant to phagocytosis by cells of the immune system in eukaryotic hosts, meaning they can evade the immune system and thus provide antibiotic resistance. Therefore, expression of annexins in pathogenic bacteria, by promoting the breakdown of the bacterial filaments, can serve as an adjuvant to enhance the efficacy of antibiotics, including beta-lactam antibiotics.

One embodiment provides a composition comprising an annexin polypeptide or subunit thereof and an antibiotic.

One embodiment provides a method to breakdown bacterial filaments in bacteria comprising contacting or expressing an annexin or subunit thereof in said bacteria so that the bacterial filaments are shorter than those of a bacterium that are not contacted with or expressing said annexin or subunit thereof. In one embodiment, the bacteria are exposed to an external stress. In one embodiment, the external stress is exposure to an antibiotic, malnutrition, chemical, osmolarity change, pH, change temperature change or combination thereof. In one embodiment the stress comprises an antibiotic, such as beta lactam. In one embodiment, the bacteria are pathogenic bacteria. In one embodiment, the annexin comprises an annexin A4. In another embodiment, the subunit comprises a single annexin repeat.

One embodiment provides a method to increase phagocytosis of bacteria that have been exposed to an external stress comprising contacting or expressing an annexin or subunit thereof in said bacteria thereby rendering the bacteria more susceptible to phagocytosis as compared to bacteria that have been exposed to an external stress and are not contacted with or expressing said annexin or subunit thereof. In one embodiment, the external stress is exposure to an antibiotic, malnutrition, chemical, osmolarity, pH, temperature or combination thereof. In one embodiment, the stress comprises an antibiotic.

Another embodiment provides a method to kill bacteria comprising contacting or expressing an annexin polypeptide or subunit thereof in said bacteria in combination with an antibiotic. In one embodiment, the contacting comprises administering said annexin polypeptide or subunit thereof and an antibiotic to a subject in need thereof. In another embodiment, a polynucleotide coding for the annexin polypeptide or subunit thereof is administered in a vector. In one embodiment, the bacteria are pathogenic. In one embodiment, the antibiotic is a beta lactam, including ampicillin, piperacillin, and cephalexin. In one embodiment, the subunit comprises a single annexin repeat. In one embodiment, the antibiotic and annexin polypeptide or a subunit thereof are administered sequentially. In another embodiment, the antibiotic and annexin polypeptide or a subunit thereof are administered concurrently.

One embodiment provides a method to increase the efficacy of an antibiotic comprising contacting or expressing annexin in bacteria wherein in said bacteria are also exposed to said antibiotic, wherein the efficacy of the antibiotic is increased as compared to bacteria not expressing or contacted with said annexin. In one embodiment, the contacting comprises administering said annexin polypeptide or subunit thereof and an antibiotic to a subject in need thereof. In another embodiment, a polynucleotide coding for the annexin polypeptide or subunit thereof is administered in a vector/as part of a vector. In one embodiment, the bacteria are pathogenic. In another embodiment, the antibiotic is a beta lactam, such as ampicillin, piperacillin, and cephalexin.

One embodiment provides a method to inhibit or reduce the development of antibiotic-resistant bacteria comprising contacting or expressing annexin in said bacteria wherein said bacteria are also exposed to said antibiotic, wherein the bacteria are inhibited as compared to bacteria not expressing or contacted with said annexin. In one embodiment, the contacting comprises administering said annexin polypeptide or subunit thereof and an antibiotic to a subject in need thereof. In one embodiment, a polynucleotide coding for the annexin polypeptide or subunit thereof is administered in a vector. In one embodiment, the antibiotic is a beta lactam.

One embodiment provides a method to breakdown/disrupt bacterial filaments in bacteria comprising contacting or expressing annexin in said bacteria. Another embodiment provides a method to prevent biofilm formation comprising contacting or expressing annexin in bacteria that can make biofilms, thereby preventing said bacteria from making biofilms.

### BRIEF DESCRIPTION OF THE DRAWINGS

FIGS. 1A-1F: Dark field light micrographs of E. coli grown in ampicillin (200 μg/ml) and expressing wild type or mutant annexin A4. 1A: Ø, control plasmid with no insert; 1B: WT wild type annexin A4; 1C: DE1234 mutant annexin A4; 1D: DE12 mutant annexin A4; 1E: DE34 mutant annexin A4; 1F: DE234 mutant annexin A4. Calibration bar on the bottom right of each panel=50 μm

FIGS. 2A-2F: Dark field light micrographs of E. coli grown in ampicillin (200 μg/ml) plus piperacillin (4 μg/ml) and expressing wild type or mutant annexin A4. 2A: Ø, control plasmid with no insert; 2B: WT wild type annexin A4; 2C: DE1234 mutant annexin A4; 2D: DE12 mutant annexin A4; 2E: DE34 mutant annexin A4; 2F: DE234 mutant annexin A4. Calibration bar on the bottom right of each panel=50 μm.

FIGS. 3A-3F: Dark field light micrographs of E. coli grown in ampicillin (200μg/ml) plus cephalexin (25 μg/ml) and expressing wild type or mutant annexin A4. 3A: Ø control plasmid with no insert; 3 hr 20 min, time after initial inoculation of the culture; 3B: WT wild type annexin A4; 3 hr 20 min, time after initial inoculation of the culture; 3C: DE234 mutant annexin A4; 3 hr 20 min, time after initial inoculation of the culture; 3D: DE1234 mutant annexin A4; 3 hr 20 min, time after initial inoculation of the culture; 3E: DE234 mutant annexin A4; 1 hr after ceph/10 (this is the culture from FIG. 3C after diluting the cephalexin 10-fold and continuing the incubation for 1 hr); 3F: DE234 mutant annexin A4; 2 hr after ceph/10 (this is the diluted culture from FIG. 3E after waiting another hour during which cytokinesis continues-see text for additional details). Calibration bar on the bottom right of each panel=50 μm.

FIGS. 4A-4D: Electron micrographs of E. coli grown in ampicillin (200 μg/ml) plus piperacillin (4 μg/ml) and expressing wild type or mutant annexin A4. 4A: Ø control plasmid with no insert; 4B: WT wild type annexin A4; 4C: DE234 mutant annexin A4; 4D: DE1234 mutant annexin A4; Calibration bar in each panel=2.5 μm.

### DETAILED DESCRIPTION OF THE INVENTION

Treatment of bacteria with antibiotics, such as beta-lactam antibiotics, can impair the process of cytokinesis, the final step in cell division, leading to the formation of a filamentous form of the bacteria. External stress (a stressor that can induce a stress response in bacteria can be any condition outside of the ideal conditions for survival) can be an antibiotics, malnutrition (e.g., nutrient deprivation), chemical exposure (e.g., reactive oxygen or chlorine species), exposure to oxidants, hypo/hyper-osmolarity, pH changes (e.g., extreme pHs), temperature changes (such as sudden increases or decreases in temperature), heat, and/or cold, which can also lead to the formation of filamentous bacteria (bacteria with longer filaments than similar bacteria not undergoing stress). As an example, the expression of a mammalian calcium-dependent, membrane-binding protein, bovine annexin A4, in E. coli was found to reverse the inhibitory effects on cytokinesis of antibiotics, including the beta-lactam ampicillin, piperacillin, and cephalexin. This novel activity of the annexin was blocked by mutation of calcium binding sites in the annexin, indicating roles for calcium binding to the annexin and the binding of the annexin to membranes in restoring cytokinesis. The filamentous form of the bacteria has been reported to be more resistant to phagocytosis by cells of the immune system in eukaryotic hosts. Therefore, expression of exposure to annexins in bacteria, by promoting the breakdown of the bacterial filaments, can serve as an adjuvant to enhance the efficacy of antibiotics and breakdown of bacterial filaments in bacteria exposed to a stressor in general.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R.J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

A “vector” or “delivery” vehicle refers to a macromolecule or association of macromolecules that comprises or associates with a polynucleotide or polypeptide, and which can be used to mediate delivery of the polynucleotide or polypeptide to a cell or intercellular space, either in vitro or in vivo (e.g., a “vector” is a replicon, such as plasmid, phage or cosmid, to which another DNA segment may be attached so as to bring about the replication of the attached segment.). Illustrative vectors include, for example, plasmids, viral vectors, liposomes, nanoparticles, bacteriophage or microparticles and other delivery vehicles. In one embodiment, a polynucleotide to be delivered, sometimes referred to as a “target polynucleotide” or “transgene,” may comprise a coding sequence of interest in gene therapy (such as a gene encoding a protein of therapeutic interest), a coding sequence of interest and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots. Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle, such as a bacteriophage, is one that comprises a polynucleotide component which is capable of delivering into a cell (e.g., bacterial cell) for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double-and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “replicon” is any genetic element (e.g., plasmid, chromosome, virus) that functions as an autonomous unit of DNA replication in vivo; i.e., capable of replication under its own control.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A polyadenylation signal and transcription termination sequence will usually be located 3′to the coding sequence.

Transcriptional and translational control sequences are DNA regulatory sequences, such as promoters, enhancers, polyadenylation signals, terminators, and the like, that provide for the expression of a coding sequence in a host cell.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators) and may be comprised of sequence-specific termination sequences or sequence-non-specific terminators or both. A variety of such terminator sequences are known in the art.

“Host cells,” “cell lines,” “cell cultures,” “packaging cell line” and other such terms denote higher eukaryotic cells, such as mammalian cells including human cells, useful in the present invention. e.g., to produce recombinant virus or recombinant polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “expression vector” is a vector comprising a region which encodes a gene product of interest and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. For example, a 2-fold enrichment, 10-fold enrichment, 100-fold enrichment, or a 1000-fold enrichment.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells or is otherwise flanked by a different nucleic acid sequence than that found in nature, e.g., an expression cassette which links a promoter from one gene to an open reading frame for a gene product from a different gene.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 82%, 85%, 87%, 90%, 92%, 95%, 97% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or 1) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

Mutations can be made in the amino acid sequences, or in the nucleic acid sequences encoding annexin A4 or in active fragments or truncations thereof, such that a particular codon is changed to a codon which codes for a different amino acid, an amino acid is substituted for another amino acid, or one or more amino acids are deleted. Such a mutation is generally made by making the fewest amino acid or nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, such as atherosclerosis, plaque buildup, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate. Such as treating subject, such as a mammal, such as a human or companion animal, such as a dog or cat, or livestock, such as pig, cow, horse, or chicken.

The terms “inhibit,” “inhibiting,” and “inhibition” refer to the slowing, halting, or reversing the growth or progression of a disease, infection, condition, group of cells, protein or its expression. The inhibition or increasing can be greater than about 20%, 40%, 60%, 80%, 90%, 95%, or 99%, for example, compared to the growth or progression that occurs in the absence or presence of the treatment or contacting.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this invention.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

The term “comprise” generally used in the sense of include, that is to say permitting the presence of one or more features or components. The term “consisting essentially of’ refers to a product, particularly a peptide sequence, of a defined number of residues which is not covalently attached to a larger product. In the case of the peptide of the invention hereof, those of skill in the art will appreciate that minor modifications to the N- or C-terminal of the peptide may however be contemplated, such as the chemical modification of the terminal to add a protecting group or the like, e.g., the amidation of the C-terminus.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

### Annexin

Annexins are a widely distributed multigene superfamily of structurally related calcium-dependent membrane-binding proteins that show a characteristic tetrad structure of homologous internal repeats. They are expressed in many organisms from protists to higher eukaryotes, including plants. The expression level and tissue distribution of vertebrate annexins span a broad range, from abundant and ubiquitous (annexins A1, A2, A4, A5, A6, A7, A11) to selective (such as annexin A3 in neutrophils and annexin A8 in the placenta and skin) or restrictive (such as annexin A9 in the tongue, annexin A10 in the stomach and annexin A13 in the small intestine). A review of annexins is provided by SE Moss and RO Morgan in an article titled “The annexins” (2004. Genome Biol. 2004;5(4):219).

In humans, the annexins are found inside the cell. However, some annexins (Annexin A1, Annexin A2, and Annexin A5) can be secreted from the cytoplasm to outside cellular environments, such as blood.

As of 2002, 160 annexin proteins have been identified in 65 different species (Gerke V, Moss S (2002). “Annexins: form structure to function”. Physiol. Rev. 82 (2): 331-71). The criteria that a protein has to meet to be classified as an annexin are it has to be capable of binding negatively charged phospholipids in a calcium dependent manner and must contain a 70 amino acid repeat sequence called an annexin repeat.

The basic structure of an annexin is composed of two major domains. The first is located at the COOH terminal and is called the “core” region. The second is located at the NH2 terminal and is called the “head” region. The core region consists of an alpha helical disk. The convex side of this disk has type 2 calcium-binding sites. They play a role for allowing interaction with the phospholipids at the plasma membrane. The N terminal region is located on the concave side of the core region and plays a role in providing a binding site for cytoplasmic proteins. In some annexins it can become phosphorylated and can cause affinity changes for calcium in the core region or alter cytoplasmic protein interaction.

The 310 amino acid annexin core has four annexin repeats, each composed of 5 alpha-helices. The exception is annexin A-VI that has two annexin core domains connected by a flexible linker. The four annexin repeats produce a curved protein and allow functional differences based on the structure of the curve. The concave side of the annexin core interacts with the N-terminus and cytosolic second messengers, while the convex side of the annexin contains calcium binding sites. Each annexin core contains one type II, also known as an annexin type, calcium binding site; these binding sites are the typical location of ionic membrane interactions. However, other methods of membrane connections are possible.

The diverse structure of the N-terminus confers specificity to annexin intracellular signaling. In annexins the N-terminus is thought to sit inside the concave side of the annexin core and folds separately from the rest of the protein. The structure of this region can be divided into two broad categories, short and long N-termini. A short N-terminus, as seen in A-III, can consist of 16 or less amino acids and travels along the concave protein core interacting via hydrogen bonds. Short N-termini are thought to stabilize the annexin complex in order to increase calcium binding and can be the sites for post-translational modifications. Long N-termini can contain up to 40 residues and have a more complex role in annexin signaling. For example, in A-I the N-terminus folds into an amphipathic alpha-helix and inserts into the protein core, displacing helix D of annexin repeat III. However, when calcium binds, the N-terminus is pushed from the annexin core by conformational changes within the protein. Therefore, the N-terminus can interact with other proteins, notably the S-100 protein family, and includes phosphorylation sites which allow for further signaling. A-II can also use its long N-terminal to form a heterotrimer between a S100 protein and two peripheral annexins. The structural diversity of annexins is the grounds for the functional range of these complex, intracellular messengers.

Annexin A4 is a protein that in humans is encoded by the ANXA4 gene. Annexin IV (ANX4) belongs to the annexin family of calcium-dependent phospholipid binding proteins. Although their functions are still not clearly defined, several members of the annexin family have been implicated in membrane-related events along exocytotic and endocytotic pathways. ANX4 has 45 to 59% identity with other members of its family and shares a similar size and exon-intron organization. Isolated from human placenta, ANX4 encodes a protein that has possible interactions with ATP and has in vitro anticoagulant activity and also inhibits phospholipase A2 activity.

### Examples of Annexin

Annexin A1, also known as lipocortin I, is a protein that is encoded by the ANXA1 gene in humans (mRNA: NM_000700; protein NP_000691).

| 1 | agtgtgaaat cttcagagaa gaatttctct ttagttcttt gcaagaaggt agagataaag |
|---|---|
| 61 | acactttttc aaaaatggca atggtatcag aattcctcaa gcaggcctgg tttattgaaa |
| 121 | atgaagagca ggaatatgtt caaactgtga agtcatccaa aggtggtccc ggatcagcgg |
| 181 | tgagccccta tcctaccttc aatccatcct cggatgtcgc tgccttgcat aaggccataa |
| 241 | tggttaaagg tgtggatgaa gcaaccatca ttgacattct aactaagcga aacaatgcac |
| 301 | agcgtcaaca gatcaaagca gcatatctcc aggaaacagg aaagcccctg gatgaaacac |
| 361 | tgaagaaagc ccttacaggt caccttgagg aggttgtttt agctctgcta aaaactccag |
| 421 | cgcaatttga tgctgatgaa cttcgtgctg ccatgaaggg ccttggaact gatgaagata |
| 481 | ctctaattga gattttggca tcaagaacta acaaagaaat cagagacatt aacagggtct |
| 541 | acagagagga actgaagaga gatctggcca aagacataac ctcagacaca tctggagatt |
| 601 | ttcggaacgc tttgctttct cttgctaagg gtgaccgatc tgaggacttt ggtgtgaatg |
| 661 | aagacttggc tgattcagat gccagggcct tgtatgaagc aggagaaagg agaaagggga |
| 721 | cagacgtaaa cgtgttcaat accatcctta ccaccagaag ctatccacaa cttcgcagag |
| 781 | tgtttcagaa atacaccaag tacagtaagc atgacatgaa caaagttctg gacctggagt |
| 841 | tgaaaggtga cattgagaaa tgcctcacag ctatcgtgaa gtgcgccaca agcaaaccag |
| 901 | ctttctttgc agagaagctt catcaagcca tgaaaggtgt tggaactcgc cataaggcat |
| 961 | tgatcaggat tatggtttcc cgttctgaaa ttgacatgaa tgatatcaaa gcattctatc |
| 1021 | agaagatgta tggtatctcc ctttgccaag ccatcctgga tgaaaccaaa ggagattatg |
| 1081 | agaaaatcct ggtggctctt tgtggaggaa actaaacatt cccttgatgg tctcaagcta |
| 1141 | tgatcagaag actttaatta tatattttca tcctataagc ttaaatagga aagtttcttc |
| 1201 | aacaggatta cagtgtagct acctacatgc tgaaaaatat agcctttaaa tcatttttat |
| 1261 | attataactc tgtataatag agataagtcc attttttaaa aatgttttcc ccaaaccata |
| 1321 | aaaccctata caagttgttc tagtaacaat acatgagaaa gatgtctatg tagctgaaaa |
| 1381 | taaaatgacg tcacaagaca a |
| (SEQ ID NO: 1 or 75, 80, 85, 90, 95, 97, 98 or 100% identical thereto.) | |
| 1 | mamvseflkq awfieneeqe yvqtvksskg gpgsavspyp tfnpssdvaa lhkaimvkgv |
| 61 | deatiidilt krnnaqrqqi kaaylqetgk pldetlkkal tghleevvla llktpaqfda |
| 121 | delraamkgl gtdedtliei lasrtnkeir dinrvyreel krdlakdits dtsgdfrnal |
| 181 | lslakgdrse dfgvnedlad sdaralyeag errkgtdvnv fntilttrsy pqlrrvfqky |
| 241 | tkyskhdmnk vldlelkgdi ekcltaivkc atskpaffae klhqamkgvg trhkalirim |
| 301 | vsrseidmnd ikafyqkmyg islcqailde tkgdyekilv alcggn |

(SEQ ID NO: 2) or 75, 80, 85, 90, 95, 97, 98 or 100% identical thereto.)

Annexin A2 also known as annexin II is a protein that in humans is encoded by the ANXA2 gene (mRNA NM_001002857; NM_001002858; NM_001136015; NM_004039; protein NP_001002857; NP_001002858; NP_001129487; NP_004030).

| 1 | gctcagcatt tggggacgct ctcagctctc ggcgcacggc ccaggttatc ttgtagcata |
|---|---|
| 61 | gcaacttcgg atttcactct acccggagag tttcccgctt ggttgaacac attggcctca |
| 121 | ggaagcttcc ttcaaaatgt ctactgttca cgaaatcctg tgcaagctca gcttggaggg |
| 181 | tgatcactct acacccccaa gtgcatatgg gtctgtcaaa gcctatacta actttgatgc |
| 241 | tgagcgggat gctttgaaca ttgaaacagc catcaagacc aaaggtgtgg atgaggtcac |
| 301 | cattgtcaac attttgacca accgcagcaa tgcacagaga caggatattg ccttcgccta |
| 361 | ccagagaagg accaaaaagg aacttgcatc agcactgaag tcagccttat ctggccacct |
| 421 | ggagacggtg attttgggcc tattgaagac acctgctcag tatgacgctt ctgagctaaa |
| 481 | agcttccatg aaggggctgg gaaccgacga ggactctctc attgagatca tctgctccag |
| 541 | aaccaaccag gagctgcagg aaattaacag agtctacaag gaaatgtaca agactgatct |
| 601 | ggagaaggac attatttcgg acacatctgg tgacttccgc aagctgatgg ttgccctggc |
| 661 | aaagggtaga agagcagagg atggctctgt cattgattat gaactgattg accaagatgc |
| 721 | tcgggatctc tatgacgctg gagtgaagag gaaaggaact gatgttccca agtggatcag |
| 781 | catcatgacc gagcggagcg tgccccacct ccagaaagta tttgataggt acaagagtta |
| 841 | cagcccttat gacatgttgg aaagcatcag gaaagaggtt aaaggagacc tggaaaatgc |
| 901 | tttcctgaac ctggttcagt gcattcagaa caagcccctg tattttgctg atcggctgta |
| 961 | tgactccatg aagggcaagg ggacgcgaga taaggtcctg atcagaatca tggtctcccg |
| 1021 | cagtgaagtg gacatgttga aaattaggtc tgaattcaag agaaagtacg gcaagtccct |
| 1081 | gtactattat atccagcaag acactaaggg cgactaccag aaagcgctgc tgtacctgtg |
| 1141 | tggtggagat gactgaagcc cgacacggcc tgagcgtcca gaaatggtgc tcaccatgct |
| 1201 | tccagctaac aggtctagaa aaccagcttg cgaataacag tccccgtggc catccctgtg |
| 1261 | agggtgacgt tagcattacc cccaacctca ttttagttgc ctaagcattg cctggccttc |
| 1321 | ctgtctagtc tctcctgtaa gccaaagaaa tgaacattcc aaggagttgg aagtgaagtc |
| 1381 | tatgatgtga aacactttgc ctcctgtgta ctgtgtcata aacagatgaa taaactgaat |
| 1441 | ttgtacttta gaaacacgta ctttgtggcc ctgctttcaa ctgaattgtt tgaaaattaa |
| 1501 | acgtgcttgg ggttcagctg gtgaggctgt ccctgtagga agaaagctct gggactgagc |
| 1561 | tgtacagtat ggttgcccct atccaagtgt cgctatttaa gttaaattta aatgaaataa |
| 1621 | aataaaataa aatca |
| (SEQ ID NO: 3 or 75, 80, 85, 90, 95, 97, 98 or 100% identical thereto). | |
| 1 | mstvheilck lslegdhstp psaygsvkay tnfdaerdal nietaiktkg vdevtivnil |
| 61 | tnrsnaqrqd iafayqrrtk kelasalksa lsghletvil gllktpaqyd aselkasmkg |
| 121 | lgtdedslie iicsrtnqel qeinrvykem yktdlekdii sdtsgdfrkl mvalakgrra |
| 181 | edgsvidyel idqdardlyd agvkrkgtdv pkwisimter svphlqkvfd ryksyspydm |
| 241 | lesirkevkg dlenaflnlv qciqnkplyf adrlydsmkg kgtrdkvlir imvsrsevdm |
| 301 | lkirsefkrk ygkslyyyiq qdtkgdyqka llylcggdd |

(SEQ ID NO: 4 or 75, 80, 85, 90, 95, 97, 98 or 100% identical thereto).

Annexin A3 is a protein that in humans is encoded by the ANXA3 gene (mRNA NM_005139; protein NP_005130).

| 1 | agcgcggagc acctgcgccc gcggctgaca ccttcgctcg cagtttgttc gcagtttact |
|---|---|
| 61 | cgcacaccag tttcccccac cgcgctttgg attagtgtga tctcagctca aggcaaaggt |
| 121 | gggatatcat ggcatctatc tgggttggac accgaggaac agtaagagat tatccagact |
| 181 | ttagcccatc agtggatgct gaagctattc agaaagcaat cagaggaatt ggaactgatg |
| 241 | agaaaatgct catcagcatt ctgactgaga ggtcaaatgc acagcggcag ctgattgtta |
| 301 | aggaatatca agcagcatat ggaaaggagc tgaaagatga cttgaagggt gatctctctg |
| 361 | gccactttga gcatctcatg gtggccctag tgactccacc agcagtcttt gatgcaaagc |
| 421 | agctaaagaa atccatgaag ggcgcgggaa caaacgaaga tgccttgatt gaaatcttaa |
| 481 | ctaccaggac aagcaggcaa atgaaggata tctctcaagc ctattataca gtatacaaga |
| 541 | agagtcttgg agatgacatt agttccgaaa catctggtga cttccggaaa gctctgttga |
| 601 | ctttggcaga tggcagaaga gatgaaagtc tgaaagtgga tgagcatctg gccaaacaag |
| 661 | atgcccagat tctctataaa gctggtgaga acagatgggg cacggatgaa gacaaattca |
| 721 | ctgagatcct gtgtttaagg agctttcctc aattaaaact aacatttgat gaatacagaa |
| 781 | atatcagcca aaaggacatt gtggacagca taaaaggaga attatctggg cattttgaag |
| 841 | acttactgtt ggccatagtt aattgtgtga ggaacacgcc ggccttttta gccgaaagac |
| 901 | tgcatcgagc cttgaagggt attggaactg atgagtttac tctgaaccga ataatggtgt |
| 961 | ccagatcaga aattgacctt ttggacattc gaacagagtt caagaagcat tatggctatt |
| 1021 | ccctatattc agcaattaaa tcggatactt ctggagacta tgaaatcaca ctcttaaaaa |
| 1081 | tctgtggtgg agatgactga accaagaaga taatctccaa aggtccacga tgggctttcc |
| 1141 | caacagctcc accttacttc ttctcatact atttaagaga acaagcaaat ataaacagca |
| 1201 | acttgtgttc ctaacaggaa ttttcattgt tctataacaa caacaacaaa agcgattatt |
| 1261 | attttagagc atctcattta taatgtagca gctcataaat gaaattgaaa atggtattaa |
| 1321 | agatctgcaa ctactatcca acttatattt ctgctttcaa agttaagaat ctttatagtt |
| 1381 | ctactccatt aaatataaag caagataata aaaattgttg cttttgttaa aa |
| (SEQ ID NO: 5 or 75, 80, 85, 90, 95, 97, 98 or 100% identical thereto). | |
| 1 | masiwvghrg tvrdypdfsp svdaeaiqka irgigtdekm lisiltersn aqrqlivkey |
| 61 | qaaygkelkd dlkgdlsghf ehlmvalvtp pavfdakqlk ksmkgagtne dalieilttr |
| 121 | tsrqmkdisq ayytvykksl gddissetsg dfrkalltla dgrrdeslkv dehlakqdaq |
| 181 | ilykagenrw gtdedkftei lclrsfpqlk ltfdeyrnis qkdivdsikg elsghfedll |
| 241 | laivncvrnt paflaerlhr alkgigtdef tlnrimvsrs eidlldirte fkkhygysly |
| 301 | saiksdtsgd yeitllkicg gdd |

(SEQ ID NO: 6 or 75, 80, 85, 90, 95, 97, 98 or 100% identical thereto).

In one embodiment, human Annexin A4 is used. Annexin A4 (human mRNA accession numbers: NM_001153; NM_001320698; NM_001320700; NM_001320702; and M_001365496; human protein accession numbers: NP_001144; NP_001307627; NP_001307629; NP_001307631; and NP_001352425). As an example, human mRNA sequence for Annexin A4 can be:

| | |
|---|---|
| 1 | gtgacctccg cagccgcaga ggaggagcgc agcccggcct cgaagaactt ctgcttgggt |
| 61 | ggctgaactc tgatcttgac ctagagtcat ggccatggca accaaaggag gtactgtcaa |
| 121 | agctgcttca ggattcaatg ccatggaaga tgcccagacc ctgaggaagg ccatgaaagg |
| 181 | gctcggcacc gatgaagacg ccattattag cgtccttgcc taccgcaaca ccgcccagcg |
| 241 | ccaggagatc aggacagcct acaagagcac catcggcagg gacttgatag acgacctgaa |
| 301 | gtcagaactg agtggcaact tcgagcaggt gattgtgggg atgatgacgc ccacggtgct |
| 361 | gtatgacgtg caagagctgc gaagggccat gaagggagcc ggcactgatg agggctgcct |
| 421 | aattgagatc ctggcctccc ggacccctga ggagatccgg cgcataagcc aaacctacca |
| 481 | gcagcaatat ggacggagcc ttgaagatga cattcgctct gacacatcgt tcatgttcca |
| 541 | gcgagtgctg gtgtctctgt cagctggtgg gagggatgaa ggaaattatc tggacgatgc |
| 601 | tctcgtgaga caggatgccc aggacctgta tgaggctgga gagaagaaat gggggacaga |
| 661 | tgaggtgaaa tttctaactg ttctctgttc ccggaaccga aatcacctgt tgcatgtgtt |
| 721 | tgatgaatac aaaaggatat cacagaagga tattgaacag agtattaaat ctgaaacatc |
| 781 | tggtagcttt gaagatgctc tgctggctat agtaaagtgc atgaggaaca aatctgcata |
| 841 | ttttgctgaa aagctctata aatcgatgaa gggcttgggc accgatgata acaccctcat |
| 901 | cagagtgatg gtttctcgag cagaaattga catgttggat atccgggcac acttcaagag |
| 961 | actctatgga aagtctctgt actcgttcat caagggtgac acatctggag actacaggaa |
| 1021 | agtactgctt gttctctgtg gaggagatga ttaaaataaa aatcccagaa ggacaggagg |
| 1081 | attctcaaca ctttgaattt ttttaacttc atttttctac actgctatta tcattatctc |
| 1141 | agaatgctta tttccaatta aaacgcctac agctgcctcc tagaatatag actgtctgta |
| 1201 | ttattattca cctataatta gtcattatga tgctttaaag ctgtacttgc atttcaaagc |
| 1261 | ttataagata taaatggaga ttttaaagta gaaataaata tgtattccat gtttttaaaa |
| 1321 | gattactttc tactttgtgt ttcacagaca ttgaatatat taaattattc catattttct |
| 1381 | tttcagtgaa aaatttttta aatggaagac tgttctaaaa tcactttttt ccctaatcca |
| 1441 | atttttagag tggctagtag tttcttcatt tgaaattgta agcatccggt cagtaagaat |
| 1501 | gcccatccag ttttctatat ttcatagtca aagccttgaa agcatctaca aatctctttt |
| 1561 | tttaggtttt gtccatagca tcagttgatc cttactaagt ttttcatggg agacttcctt |
| 1621 | catcacatct tatgttgaaa tcactttctg tagtcaaagt ataccaaaac caatttatct |
| 1681 | gaactaaatt ctaaagtatg gttatacaaa ccatatacat ctggttacca aacataaatg |
| 1741 | ctgaacattc catattatta tagttaatgt cttaatccag cttgcaagtg aatggaaaaa |
| 1801 | aaaataagct tcaaactagg tattctggga atgatgtaat gctctgaatt tagtatgata |
| 1861 | taaagaaaac ttttttgtgc taaaaatact ttttaaaatc aattttgttg attgtagtaa |
| 1921 | tttctatttg cactgtgcct ttcaactcca gaaacattct gaagatgtac ttggatttaa |
| 1981 | ttaaaaagtt cactttgtaa gaacgtggaa aaataatttt aatttaaaaa tggtgttttt |
| 2041 | aggccggggg cgggggctca cgccagtaat cccaacactt tgggaggcca aggcgggtgg |
| 2101 | atcacctaag gtcaggagtt caagactagc ctggccaaca tggagaaact gcatctctac |
| 2161 | taaaaatata aaaattagcc gggtgtggtg gctggtgcct gtaatcccag ccactcggag |
| 2221 | gctgagtcag ggagaactgc ttgaacccag gaggcaggag gcaaaggttg cagtgagccg |
| 2281 | agatcacgcc agcctgggcg acagagcgag aatccatcta aaaaaaaaaa aaaaaaaagt |
| 2341 | gtctttaaag tgaggtatag tctttctctg atccactttt caccttctga ggtttttcat |
| 2401 | cttggcccct gaaaggagct atttttgaag gacttgtgtt actcagtttc tacaggaatt |
| 2461 | acaagataag aaaaaaaaaa tcatatttag tcttatgcgt gcctactggc taatgttcac |
| 2521 | atatgccaaa cactactcaa taacataaaa taatgtatga acttattctc tggaaatgag |
| 2581 | tgatgccctc tgctctaagt agaccattta tattaaatat cataaatgta taaaggacat |
| 2641 | tcatattctt a |

(SEQ ID NO: 7 or 75, 80, 85, 90, 95, 97, 98 or 100% identical thereto).

As an example, human protein sequence for Annexin A4 can be:

| | |
|---|---|
| 1 | mamatkggtv kaasgfname daqtlrkamk glgtdedaii svlayrntaq rqeirtayks |
| 61 | tigrdliddl kselsgnfeq vivgmmtptv lydvqelrra mkgagtdegc lieilasrtp |
| 121 | eeirrisqty qqqygrsled dirsdtsfmf qrvlvslsag grdegnyldd alvrqdaqdl |
| 181 | yeagekkwgt devkfltvlc srnrnhllhv fdeykrisqk dieqsikset sgsfedalla |
| 241 | ivkcmrnksa yfaeklyksm kglgtddntl irvmvsraei dmldirahfk rlygkslysf |
| 301 | ikgdtsgdyr kvllvlcggd d |

(SEQ ID NO: 8 or 75, 80, 85, 90, 95, 97, 98 or 100% identical thereto).

Annexin A5 (or annexin V) is a cellular protein in the annexin group (mRNA NM_001154; protein NP_001145).

| 1 | agtctaggtg cagctgccgg atccttcagc gtctgcatct cggcgtcgcc ccgcgtaccg |
|---|---|
| 61 | tcgcccggct ctccgccgct ctcccggggt ttcggggcac ttgggtccca cagtctggtc |
| 121 | ctgcttcacc ttcccctgac ctgagtagtc gccatggcac aggttctcag aggcactgtg |
| 181 | actgacttcc ctggatttga tgagcgggct gatgcagaaa ctcttcggaa ggctatgaaa |
| 241 | ggcttgggca cagatgagga gagcatcctg actctgttga catcccgaag taatgctcag |
| 301 | cgccaggaaa tctctgcagc ttttaagact ctgtttggca gggatcttct ggatgacctg |
| 361 | aaatcagaac taactggaaa atttgaaaaa ttaattgtgg ctctgatgaa accctctcgg |
| 421 | ctttatgatg cttatgaact gaaacatgcc ttgaagggag ctggaacaaa tgaaaaagta |
| 481 | ctgacagaaa ttattgcttc aaggacacct gaagaactga gagccatcaa acaagtttat |
| 541 | gaagaagaat atggctcaag cctggaagat gacgtggtgg gggacacttc agggtactac |
| 601 | cagcggatgt tggtggttct ccttcaggct aacagagacc ctgatgctgg aattgatgaa |
| 661 | gctcaagttg aacaagatgc tcaggcttta tttcaggctg gagaacttaa atgggggaca |
| 721 | gatgaagaaa agtttatcac catctttgga acacgaagtg tgtctcattt gagaaaggtg |
| 781 | tttgacaagt acatgactat atcaggattt caaattgagg aaaccattga ccgcgagact |
| 841 | tctggcaatt tagagcaact actccttgct gttgtgaaat ctattcgaag tatacctgcc |
| 901 | taccttgcag agaccctcta ttatgctatg aagggagctg ggacagatga tcataccctc |
| 961 | atcagagtca tggtttccag gagtgagatt gatctgttta acatcaggaa ggagtttagg |
| 1021 | aagaattttg ccacctctct ttattccatg attaagggag atacatctgg ggactataag |
| 1081 | aaagctcttc tgctgctctg tggagaagat gactaacgtg tcacggggaa gagctccctg |
| 1141 | ctgtgtgcct gcaccacccc actgccttcc ttcagcacct ttagctgcat ttgtatgcca |
| 1201 | gtgcttaaca cattgcctta ttcatactag catgctcatg accaacacat acacgtcata |
| 1261 | gaagaaaata gtggtgcttc tttctgatct ctagtggaga tctctttgac tgctgtagta |
| 1321 | ctaaagtgta cttaatgtta ctaagtttaa tgcctggcca ttttccattt atatatattt |
| 1381 | tttaagaggc tagagtgctt ttagcctttt ttaaaaactc catttatatt acatttgtaa |
| 1441 | ccatgatact ttaatcagaa gcttagcctt gaaattgtga actcttggaa atgttattag |
| 1501 | tgaagttcgc aactaaacta aacctgtaaa attatgatga ttgtattcaa aagattaatg |
| 1561 | aaaaataaac atttctgtcc ccctgaatta tgtgtacatg tgtgtttaga tttattatta |
| 1621 | aatttattta acaatgtt |
| (SEQ ID NO: 9 cr 75, 80, 85, 90, 95, 97, 98 or 100% identical thereto). | |
| 1 | maqvlrgtvt dfpgfderad aetlrkamkg lgtdeesilt lltsrsnaqr qeisaafktl |
| 61 | fgrdllddlk seltgkfekl ivalmkpsrl ydayelkhal kgagtnekvl teiiasrtpe |
| 121 | elraikqvye eeygssledd vvgdtsgyyq rmlvvllqan rdpdagidea qveqdaqalf |
| 181 | qagelkwgtd eekfitifgt rsvshlrkvf dkymtisgfq ieetidrets gnleqlllav |
| 241 | vksirsipay laetlyyamk gagtddhtli rvmvsrseid lfnirkefrk nfatslysmi |
| 301 | kgdtsgdykk allllcgedd |

(SEQ ID NO: 10 or 75, 80, 85, 90, 95, 97, 98 or 100% identical thereto).

Annexin A6 is a protein that in humans is encoded by the ANXA6 gene (mRNA NM_001155; NM_001193544; NM_004033; NM_001363114; protein NP_001146; NP_001180473; NP_001350043).

| 1 | gcggttgctg ctgggctaac gggctccgat ccagcgagcg ctgcgtcctc gagtccctgc |
|---|---|
| 61 | gcccgtgcgt ccgtctgcga cccgaggcct ccgctgcgcg tggattctgc tgcgaaccgg |
| 121 | agaccatggc caaaccagca cagggtgcca agtaccgggg ctccatccat gacttcccag |
| 181 | gctttgaccc caaccaggat gccgaggctc tgtacactgc catgaagggc tttggcagtg |
| 241 | acaaggaggc catactggac ataatcacct cacggagcaa caggcagagg caggaggtct |
| 301 | gccagagcta caagtccctc tacggcaagg acctcattgc tgatttaaag tatgaattga |
| 361 | cgggcaagtt tgaacggttg attgtgggcc tgatgaggcc acctgcctat tgtgatgcca |
| 421 | aagaaattaa agatgccatc tcgggcattg gcactgatga gaagtgcctc attgagatct |
| 481 | tggcttcccg gaccaatgag cagatgcacc agctggtggc agcatacaaa gatgcctacg |
| 541 | agcgggacct ggaggctgac atcatcggcg acacctctgg ccacttccag aagatgcttg |
| 601 | tggtcctgct ccagggaacc agggaggagg atgacgtagt gagcgaggac ctggtacaac |
| 661 | aggatgtcca ggacctatac gaggcagggg aactgaaatg gggaacagat gaagcccagt |
| 721 | tcatttacat cttgggaaat cgcagcaagc agcatcttcg gttggtgttc gatgagtatc |
| 781 | tgaagaccac agggaagccg attgaagcca gcatccgagg ggagctgtct ggggactttg |
| 841 | agaagctaat gctggccgta gtgaagtgta tccggagcac cccggaatat tttgctgaaa |
| 901 | ggctcttcaa ggctatgaag ggcctgggga ctcgggacaa caccctgatc cgcatcatgg |
| 961 | tctcccgtag tgagttggac atgctcgaca ttcgggagat cttccggacc aagtatgaga |
| 1021 | agtccctcta cagcatgatc aagaatgaca cctctggcga gtacaagaag actctgctga |
| 1081 | agctgtctgg gggagatgat gatgctgctg gccagttctt cccggaggca gcgcaggtgg |
| 1141 | cctatcagat gtgggaactt agtgcagtgg cccgagtaga gctgaaggga actgtgcgcc |
| 1201 | cagccaatga cttcaaccct gacgcagatg ccaaagcgct gcggaaagcc atgaagggac |
| 1261 | tcgggactga cgaagacaca atcatcgata tcatcacgca ccgcagcaat gtccagcggc |
| 1321 | agcagatccg gcagaccttc aagtctcact ttggccggga cttaatgact gacctgaagt |
| 1381 | ctgagatctc tggagacctg gcaaggctga ttctggggct catgatgcca ccggcccatt |
| 1441 | acgatgccaa gcagttgaag aaggccatgg agggagccgg cacagatgaa aaggctctta |
| 1501 | ttgaaatcct ggccactcgg accaatgctg aaatccgggc catcaatgag gcctataagg |
| 1561 | aggactatca caagtccctg gaggatgctc tgagctcaga cacatctggc cacttcagga |
| 1621 | ggatcctcat ttctctggcc acggggcatc gtgaggaggg aggagaaaac ctggaccagg |
| 1681 | cacgggaaga tgcccaggtg gctgctgaga tcttggaaat agcagacaca cctagtggag |
| 1741 | acaaaacttc cttggagaca cgtttcatga cgatcctgtg tacccggagc tatccgcacc |
| 1801 | tccggagagt cttccaggag ttcatcaaga tgaccaacta tgacgtggag cacaccatca |
| 1861 | agaaggagat gtctggggat gtcagggatg catttgtggc cattgttcaa agtgtcaaga |
| 1921 | acaagcctct cttctttgcc gacaaacttt acaaatccat gaagggtgct ggcacagatg |
| 1981 | agaagactct gaccaggatc atggtatccc gcagtgagat tgacctgctc aacatccgga |
| 2041 | gggaattcat tgagaaatat gacaagtctc tccaccaagc cattgagggt gacacctccg |
| 2101 | gagacttcct gaaggccttg ctggctctct gtggtggtga ggactagggc cacagctttg |
| 2161 | gcgggcactt ctgccaagaa atggttatca gcaccagccg ccatggccaa gcctgattgt |
| 2221 | tccagctcca gagactaagg aaggggcagg ggtgggggga ggggttgggt tgggctctta |
| 2281 | tcttcagtgg agcttaggaa acgctcccac tcccacgggc catcgagggc ccagcacggc |
| 2341 | tgagcggctg aaaaaccgta gccatagatc ctgtccacct ccactcccct ctgaccctca |
| 2401 | ggctttccca gcttcctccc cttgctacag cctctgccct ggtttgggct atgtcagatc |
| 2461 | caaaaacatc ctgaacctct gtctgtaaaa tgagtagtgt ctgtactttg aatgaggggg |
| 2521 | ttggtggcag gggccagttg aatgtgctgg gcggggtggt gggaaggata gtaaatgtgc |
| 2581 | tggggcaaac tgacaaatct tcccatccat ttcaccaccc atctccatcc aggccgcgct |
| 2641 | agagtactgg accaggaatt tggatgcctg ggttcaaatc tgcatctgcc atgcacttgt |
| 2701 | ttctgacctt aggccagccc ctttccctcc ctgagtctct attttcttat ctacaatgag |
| 2761 | acagttggac aaaaaaatct tggcttccct tctaacatta acttcctaaa gtatgcctcc |
| 2821 | gattcattcc cttgacactt tttatttcta aggaagaaat aaaaagagat acacaaacac |
| 2881 | ataaacaca |
| (SEQ ID NO: 11 or 75, 80, 85, 90, 95, 97, 98 or 100% identical thereto). | |
| 1 | makpaqgaky rgsihdfpgf dpnqdaealy tamkgfgsdk eaildiitsr snrqrqevcq |
| 61 | sykslygkdl iadlkyeltg kferlivglm rppaycdake ikdaisgigt dekclieila |
| 121 | srtneqmhql vaaykdayer dleadiigdt sghfqkmlvv llqgtreedd vvsedlvqqd |
| 181 | vqdlyeagel kwgtdeaqfi yilgnrskqh lrlvfdeylk ttgkpieasi rgelsgdfek |
| 241 | lmlavvkcir stpeyfaerl fkamkglgtr dntlirimvs rseldmldir eifrtkyeks |
| 301 | lysmikndts geykktllkl sggdddaagq ffpeaaqvay qmwelsavar velkgtvrpa |
| 361 | ndfnpdadak alrkamkglg tdedtiidii thrsnvqrqq irqtfkshfg rdlmtdlkse |
| 421 | isgdlarlil glmmppahyd akqlkkameg agtdekalie ilatrtnaei raineayked |
| 481 | yhksledals sdtsghfrri lislatghre eggenldqar edaqvaaeil eiadtpsgdk |
| 541 | tsletrfmti lctrsyphlr rvfqefikmt nydvehtikk emsgdvrdaf vaivqsvknk |
| 601 | plffadklyk smkgagtdek tltrimvsrs eidllnirre fiekydkslh qaiegdtsgd |
| 661 | flkallalcg ged |

(SEQ ID NO: 12 or 75, 80, 85, 90, 95, 97, 98 or 100% identical thereto).

Annexin A7 is a protein that in humans is encoded by the ANXA7 gene (mRNA NM_001156; NM_004034; NM_001320879; NM_001320880; protein NP_001147; NP_001307808; NP_001307809; NP_004025).

| 1 | atcttgcggg agaccgggtt gggctgtgac gctgctgctg gggtcagaat gtcataccca |
|---|---|
| 61 | ggctatcccc caacaggcta gtcataccca cccacctttc cctggatatc ctcctgcagg |
| 121 | tcttttcccc cttctggtca gtatccttat cctagtggct ttcctccaat gggaggaggt |
| 181 | gcctacccac aagtgccaag tagtggctac ccaggagctg gaggctaccc tgcgcctgga |
| 241 | ggttatccag cccctggagg ctatcctggt gccccacagc cagggggagc tccatcctat |
| 301 | cccggagttc ctccaggcca aggatttgga gtcccaccag gtggagcagg cttttctggg |
| 361 | tatccacagc caccttcaca gtcttatgga ggtggtccag cacaggttcc actacctggt |
| 421 | ggctttcctg gaggacagat gccttctcag tatcctggag gacaacctac ttaccctagt |
| 481 | cagcctgcca cagtgactca ggtcactcaa ggaactatcc gaccagctgc caacttcgat |
| 541 | gctataagag atgcagaaat tcttcgtaag gcaatgaagg gttttgggac agatgagcag |
| 601 | gcaattgtgg atgtggtggc caaccgttcc aatgatcaga ggcaaaaaat taaagcagca |
| 661 | tttaagacct cctatggcaa ggatttaatc aaagatctca aatcagagtt aagtggaaat |
| 721 | atggaagaac tgatcctggc cctcttcatg cctcctacgt attacgatgc ctggagctta |
| 781 | cggaaagcaa tgcagggagc aggaactcag gaacgtgtat tgattgagat tttgtgcaca |
| 841 | agaacaaatc aggaaatccg agaaattgtc agatgttatc agtcagaatt tggacgagac |
| 901 | cttgaaaagg acattaggtc agatacatca ggacattttg aacgtttact tgtgtccatg |
| 961 | tgccagggaa atcgtgatga gaaccagagt ataaaccacc aaatggctca ggaagatgct |
| 1021 | cagcgtctct atcaagctgg tgaggggaga ctagggaccg atgaatcttg ctttaacatg |
| 1081 | atccttgcca caagaagctt tcctcagctg agagctacca tggaggctta ttctaggatg |
| 1141 | gctaatcgag acttgttaag cagtgtgagc cgtgagtttt ccggatatgt agaaagtggt |
| 1201 | ttgaagacca tcttgcagtg tgccctgaac cgccctgcct tctttgctga gaggctctac |
| 1261 | tatgctatga aaggtgctgg cacagatgac tccaccctgg tccggattgt ggtcactcga |
| 1321 | agtgagattg accttgtaca aataaaacag atgttcgctc agatgtatca gaagactctg |
| 1381 | ggcacaatga ttgcaggtga cacgagtgga gattaccgaa gacttcttct ggctattgtg |
| 1441 | ggccagtagg agggattttt ttttttttaa tgaaaaaaaa tttctattca tagcttatcc |
| 1501 | ttcagagcaa tgacctgcat gcagcaatat caaacatcag ctaaccgaaa gagctttctg |
| 1561 | tcaaggaccg tatcagggta atgtgcttgg tttgcacatg ttgttattgc cttaattcta |
| 1621 | attttatttt gttctctaca tacaatcaat gtaaagccat atcacaatga tacagtaata |
| 1681 | ttgcaatgtt tgtaaacctt cattcttact agtttcattc taatcaagat gtcaaattga |
| 1741 | ataaaaatca cagcaatctc tgattctgtg taataatatt gaataatttt ttagaaggtt |
| 1801 | actgaaagct ctgccttccg gaatccctct aagtctgctt gatagagtgg atagtgtgtt |
| 1861 | aaaactgtgt actttaaaaa aaaattcaac ctttacatct agaataattt gcatctcatt |
| 1921 | ttgcctaaat tggttctgta ttcataaaca ctttccacat agaaaataga ttagtattac |
| 1981 | ctgtggcacc ttttaagaaa gggtcaaatg tttatatgct taagatacat agcctacttt |
| 2041 | tttttcgcag ttgttttctt tttttaaatt gagttatgac aaataaaaaa ttgcatatat |
| 2101 | ttaaggtgta caatatggtg ttttgatatc agcattcctt gtgtaatgat tccacaatta |
| 2161 | aggtcaggct aattacgtat ctgtcacctt gacatagtta ccattttttc atgtgtggtg |
| 2221 | aaaacactta agatctacta ccttagcaaa ttttaagtgt tcagtacatt attaactata |
| 2281 | gatactgtgc tctacattaa acctctagca tttattcgtt ttataactga aagtttatac |
| 2341 | cctttgacca acatctcccc attttcccca cctctcacct ggacaaccac cactgtgttt |
| 2401 | aagttcagct attttagatt ccacgtataa atggtataca ata |
| (SEQ ID NO: 13 or 75, 80, 85, 90, 95, 97, 98 or 100% identical thereto). | |
| 1 | msypgypptg yppfpgyppa gqessfppsg qypypsgfpp mgggaypqvp ssgypgaggy |
| 61 | papggypapg gypgapqpgg apsypgvppg qgfgvppgga gfsgypqpps qsygggpaqv |
| 121 | plpggfpggq mpsqypggqp typsqpatvt qvtqgtirpa anfdairdae ilrkamkgfg |
| 181 | tdeqaivdvv anrsndqrqk ikaafktsyg kdlikdlkse lsgnmeelil alfmpptyyd |
| 241 | awslrkamqg agtqervlie ilctrtnqei reivrcyqse fgrdlekdir sdtsghferl |
| 301 | lvsmcqgnrd enqsinhqma qedaqrlyqa gegrlgtdes cfnmilatrs fpqlratmea |
| 361 | ysrmanrdll ssvsrefsgy vesglktilq calnrpaffa erlyyamkga gtddstlvri |
| 421 | vvtrseidlv qikqmfaqmy qktlgtmiag dtsgdyrrll laivgq |

(SEQ ID NO: 14 or 75, 80, 85, 90, 95, 97, 98 or 100% identical thereto).

Annexin A9 is a protein that in humans is encoded by the ANXA9 gene (mRNA NM_003568; protein NP_003559).

| 1 | ctctaccagg ccacaccgga ggcagtgctc acacaggcaa gctaccaggc cacaacaacg |
|---|---|
| 61 | acacccacct cacctctggc acctctgagc atccacgtac ttgcaagaac tcttgctcac |
| 121 | atcagctaag agattgcacc tgctgaccta gagattccgg cctgtgctcc tgtgctgctg |
| 181 | agcagggcaa ccagtagcac catgtctgtg actggcggga agatggcacc gtccctcacc |
| 241 | caggagatcc tcagccacct gggcctggcc agcaagactg cagcgtgggg gaccctgggc |
| 301 | accctcagga ccttcttgaa cttcagcgtg gacaaggatg cgcagaggct actgagggcc |
| 361 | attactggcc aaggcgtgga ccgcagtgcc attgtggacg tgctgaccaa ccggagcaga |
| 421 | gagcaaaggc agctcatctc acgaaacttc caggagcgca cccaacagga cctgatgaag |
| 481 | tctctacagg cagcactttc cggcaacctg gagaggattg tgatggctct gctgcagccc |
| 541 | acagcccagt ttgacgccca ggaattgagg acagctctga aggcctcaga ttctgctgtg |
| 601 | gacgtggcca ttgaaattct tgccactcga accccacccc agctgcagga gtgcctggca |
| 661 | gtctacaaac acaatttcca ggtggaggct gtggatgaca tcacatctga gaccagtggc |
| 721 | atcttgcagg acctgctgtt ggccctggcc aaggggggcc gtgacagcta ctctggaatc |
| 781 | attgactata atctggcaga acaagatgtc caggcactgc agcgggcaga aggacctagc |
| 841 | agagaggaaa catgggtccc agtcttcacc cagcgaaatc ctgaacacct catccgagtg |
| 901 | tttgatcagt accagcggag cactgggcaa gagctggagg aggctgtcca gaaccgtttc |
| 961 | catggagatg ctcaggtggc tctgctcggc ctagcttcgg tgatcaagaa cacaccgctg |
| 1021 | tactttgctg acaaacttca tcaagccctc caggaaactg agcccaatta ccaagtcctg |
| 1081 | attcgcatcc ttatctctcg atgtgagact gaccttctga gtatcagagc tgagttcagg |
| 1141 | aagaaatttg ggaagtccct ctactcttct ctccaggatg cagtgaaagg ggattgccag |
| 1201 | tcagccctcc tggccttgtg cagggctgaa gacatgtgag acttccctgc cccaccccac |
| 1261 | atgacatccg aggatctgag atttccgtgt ttggctgaac ctgggagacc agctgggcct |
| 1321 | ccaagtagga taacccctca ctgagcacca cattctctag cttcttgttg aggctggaac |
| 1381 | tgtttcttta aaatccctta attttcccat ctcaaaatta tatctgtacc tgggtcatcc |
| 1441 | agctccttct tgggtgtggg gaaatgagtt ttctttgata gtttctgcct cactcatccc |
| 1501 | tcctgtaccc tggccagaac atctcactga tactcgaatt cttttggcaa a |
| (SEQ ID NO: 15 or 75, 80, 85, 90, 95, 97, 98 or 100% identical thereto). | |
| 1 | msvtggkmap sltqeilshl glasktaawg tlgtlrtfln fsvdkdaqrl lraitgqgvd |
| 61 | rsaivdvltn rsreqrqlis rnfqertqqd Imkslqaals gnlerivmal lqptaqfdaq |
| 121 | elrtalkasd savdvaieil atrtppqlqe clavykhnfq veavdditse tsgilqdlll |
| 181 | alakggrdsy sgiidynlae qdvqalqrae gpsreetwvp vftqrnpehl irvfdqyqrs |
| 241 | tgqeleeavq nrfhgdaqva llglasvikn tplyfadklh qalqetepny qvlirilisr |
| 301 | cetdllsira efrkkfgksl ysslqdavkg dcqsallalc raedm |

(SEQ ID NO: 16 or 75, 80, 85, 90, 95, 97, 98 or 100% identical thereto).

Annexin A11 is a protein that in humans is encoded by the ANXA11 gene (mRNA NM_001157; NM_001278407; NM_001278408; NM_001278409; NM_145868; protein NP_001148; NP_001265336; NP_001265337; NP_001265338; NP_665875).

| 1 | ggagttttcc gcccggcgct gacggctgct gcgcccgcgg ctccccagtg ccccgagtgc |
|---|---|
| 61 | cccgcgggcc ccgcgagcgg gagtgggacc cagcccctag gcagaaccca ggcgccgcgc |
| 121 | ccgggacgcc cgcggagaga gccactcccg cccacgtccc atttcgcccc tcgcgtccgg |
| 181 | agtccccgtg gccagatcta accatgagct accctggcta tcccccgccc ccaggtggct |
| 241 | acccaccagc tgcaccaggt ggtggtccct ggggaggtgc tgcctaccct cctccgccca |
| 301 | gcatgccccc catcgggctg gataacgtgg ccacctatgc ggggcagttc aaccaggact |
| 361 | atctctcggg aatggcggcc aacatgtctg ggacatttgg aggagccaac atgcccaacc |
| 421 | tgtaccctgg ggcccctggg gctggctacc caccagtgcc ccctggcggc tttgggcagc |
| 481 | ccccctctgc ccagcagcct gttcctccct atgggatgta tccaccccca ggaggaaacc |
| 541 | caccctccag gatgccctca tatccgccat acccaggggc ccctgtgccg ggccagccca |
| 601 | tgccaccccc cggacagcag cccccagggg cctaccctgg gcagccacca gtgacctacc |
| 661 | ctggtcagcc tccagtgcca ctccctgggc agcagcagcc agtgccgagc tacccaggat |
| 721 | acccggggtc tgggactgtc acccccgctg tgcccccaac ccagtttgga agccgaggca |
| 781 | ccatcactga tgctcccggc tttgaccccc tgcgagatgc cgaggtcctg cggaaggcca |
| 841 | tgaaaggctt cgggacggat gagcaggcca tcattgactg cctggggagt cgctccaaca |
| 901 | agcagcggca gcagatccta ctttccttca agacggctta cggcaaggat ttgatcaaag |
| 961 | atctgaaatc tgaactgtca ggaaactttg agaagacaat cttggctctg atgaagaccc |
| 1021 | cagtcctctt tgacatttat gagataaagg aagccatcaa gggggttggc actgatgaag |
| 1081 | cctgcctgat tgagatcctc gcttcccgca gcaatgagca catccgagaa ttaaacagag |
| 1141 | cctacaaagc agaattcaaa aagaccctgg aagaggccat tcgaagcgac acatcagggc |
| 1201 | acttccagcg gctcctcatc tctctctctc agggaaaccg tgatgaaagc acaaacgtgg |
| 1261 | acatgtcact cgcccagaga gatgcccagg agctgtatgc ggccggggag aaccgcctgg |
| 1321 | gaacagacga gtccaagttc aatgcggttc tgtgctcccg gagccgggcc cacctggtag |
| 1381 | cagttttcaa tgagtaccag agaatgacag gccgggacat tgagaagagc atctgccggg |
| 1441 | agatgtccgg ggacctggag gagggcatgc tggccgtggt gaaatgtctc aagaataccc |
| 1501 | cagccttctt tgcggagagg ctcaacaagg ccatgagggg ggcaggaaca aaggaccgga |
| 1561 | ccctgattcg catcatggtg tctcgcagcg agaccgacct cctggacatc agatcagagt |
| 1621 | ataagcggat gtacggcaag tcgctgtacc acgacatctc gggagatact tcaggggatt |
| 1681 | accggaagat tctgctgaag atctgtggtg gcaatgactg aacagtgact ggtggctcac |
| 1741 | ttctgcccac ctgccggcaa caccagtgcc aggaaaaggc caaaagaatg tctgtttcta |
| 1801 | acaaatccac aaatagcccc gagattcacc gtcctagagc ttaggcctgt cttccacccc |
| 1861 | tcctgacccg tatagtgtgc cacaggacct gggtcggtct agaactctct caggatgcct |
| 1921 | tttctacccc atccctcaca gcctcttgct gctaaaatag atgtttcatt tttctgactc |
| 1981 | atgcaatcat tcccctttgc ctgtggctaa gacttggctt catttcgtca tgtaattgta |
| 2041 | tatttttatt tggaggcata ttttcttttc ttacagtcat tgccagacag aggcatacaa |
| 2101 | gtctgtttgc tgcatacaca tttctggtga gggcgactgg gtgggtgaag caccgtgtcc |
| 2161 | tcgctgagga gagaaaggga ggcgtgcctg agaaggtagc ctgtgcatct ggtgagtgtg |
| 2221 | tcacgagctt tgttactgcc aaactcactc ctttttagaa aaaacaaaaa aaaagggcca |
| 2281 | gaaagtcatt ccttccatct tccttgcaga aaccacgaga acaaagccag ttccctgtca |
| 2341 | gtgacagggc ttcttgtaat ttgtggtatg tgccttaaac ctgaatgtct gtagccaaaa |
| 2401 | cttgtttcca cattaagagt cagccagctc tggaatggtc tggaaatgtc ttcctggtac |
| 2461 | caacttgttt tcttctgctt gattctgccc tgtggctcag aggtctggcc ttatcagcca |
| 2521 | gtgaaagttc atgtaacctt acgtagagat ttgtgtgcag gaaaccctga gcatacacta |
| 2581 | gtttgcaggg actcgtaagg acatgggaag ggaggttccc gaaatccagg caggaggccc |
| 2641 | agacacctga aaggcaaagg gatcttggtt ggttgcaggt gcagtgaagt ccactgaagg |
| 2701 | tgtggtgcga agaatgcagt ccttcaccca ggtcccagga gggaagaagg gtgtgtgcta |
| 2761 | attcctggtg cccctcggcg ggggccagag agaaggatgg ggacaaccca gagagtcaca |
| 2821 | agaccagtgc ctcccctcag ggtgcctcca ggctgaaagg ggctcctggc tctggtctct |
| 2881 | ggggaccctg tgcccgttgg ttggtggtgt gagggaagag aatccataag agagtttctg |
| 2941 | agaattatgg tgtcatgtcc agaagctaga gcttaccttg catcaggggt ctccacccac |
| 3001 | tccttttcca acctcctgcg ttgaggttta gaaaagagag aatcgactag gcactatggc |
| 3061 | tcacgcctgt aatccaagga ctttgggaag ctgaggtgag aggatcactt gagctcagga |
| 3121 | gttcaagact agcctagcca acagcgagac ccctgtctct actaaaaaat ttggccaggc |
| 3181 | gtggtggctc acggctgtaa tcccagcact ttgggaggtg aggcgggcag atcacctgag |
| 3241 | gtcaggagtt cgagacccag cctggccaac atggtgaaac cccatctcta ctaaaaatac |
| 3301 | aaaaattagc caggcatggt ggcacattcc tgtaatccca gctacacagg atgctgaggc |
| 3361 | aggagaatca cttgaaccca ggaggcagag gttgtagtga gctgagatca caccattgca |
| 3421 | cttcaacctg ggtggacaga gtgagactct gtctcaaaaa aaaaaaaaaa tttacctggc |
| 3481 | attgtagtgc attccctata gtcggctact ctggaggctg aggcaggaag atccttagag |
| 3541 | cccaagaaat tgaggccgta gtaagctgtg attacaccac tgcactccag cctggacaac |
| 3601 | agagcgagac cttgtctcaa atgagaaaaa aacaaaaaga aatgggagaa tccagagaga |
| 3661 | ctaggctaga tcaagcctgc tgggtcctgg caggagcccc agggagtagc tcatctgcag |
| 3721 | acatttgctt gaggactacc ccctaaacat aaaggaagaa tgacatccga agggtgtgga |
| 3781 | gcagccatga gctgagaact agcctggtct acctgagatt gatggcaggt cctggtcaac |
| 3841 | acgtcagctc tgcgtcagag tccatgcctc aagcccaagc tgaagcccca tccctgctgc |
| 3901 | tctcccaaga actcctctgc tagggcaggc cccttgccct tgggtgccag gtgggacctg |
| 3961 | cctgatggga tggggtgctt ggcatataca acttgccatg aactcaaggt gaccctgggg |
| 4021 | gcctcctgaa ttgtgatggg gcctagaacc aatgtgctct gatgtgacca tattctgtga |
| 4081 | cattaccttg ccctgtttac tccaaagttc ccagcctggt gcccagcagg caatattgca |
| 4141 | cctacagaca catttacttt ggtttccaaa gtgtttttag acatttgaat ttgttgccaa |
| 4201 | catttaaaca ttgagagatt tcatattttt aaaaatctgg aattctggct tctcttgaaa |
| 4261 | actcagaaat tctggcacta tggggcttgc attcctgcat ggctggagct gagttgcagc |
| 4321 | tgccccttta ggcctgtact ccttatttgc tataggctcc gtcttgtatt acactaagcc |
| 4381 | catgtcaccc atttggctcc tgcaggcctt tgggtttgag accctggtct acacacttgg |
| 4441 | agaccacctg ttgtaaagta catggatgtg ctttggtcaa ggaatagacc aaggtggata |
| 4501 | tccaggccag agtgactcag cgagtttagg tcacaggcgt atactccact tgttatataa |
| 4561 | cctgcttgtg taagttcata cttggctcaa agccactatt gtttggaaaa ggtataactg |
| 4621 | ccctgctgac gctgtacaga tgttcttggg ctcggatggg catggctcca cgtggtgtgc |
| 4681 | actagcaccc agagagagtg aagctattga cccctgtaag ggagagtgac catctggcag |
| 4741 | atagatagag gggagccagg acatggctca gcttgtgccc agagggagag ttaagccgct |
| 4801 | gaccctgtag ccagggagtg cacctgcaag catgggggtg gcaggagcca cagagctggc |
| 4861 | tgctgagagg agctgcagat ctggagaaga cagcctaggt aaaggtggac agtgtgagag |
| 4921 | ctgctgatga gatagctgct gaataaaact acattttacc tgcctatggc ccgccaggtt |
| 4981 | ttctttcagc tatcgcccat ccacccagtc ccctcgaacc tcagcatggg ctggaacctg |
| 5041 | accctgggca tgacatttgg catagttgtg gacctgacac ctgtgtttgt cctagtcctg |
| 5101 | tttctccctg ccttcctgtt cctctcgctg ccctcatggt cactcccaag agatccaacc |
| 5161 | catgttaagt atgggctgga ggactgcatg aatgcctcat gatcttccca gaggcaaagg |
| 5221 | cacctactgc cttccaaggt cagtgggagg ttgggatcaa cactgtttat tatgcttagg |
| 5281 | acaaaaaaga tagggagaaa gatgtgcaac cttacaggtc atctttctgg gatagaacac |
| 5341 | aatgggtctt ctcctgcctc ctggatatgt tagtcaaggc cagtccatgc tacacatcta |
| 5461 | gtgggggcgc tggaggaaac actattaaat agttttgcac ctgagacaga tagcctcact |
| 5521 | cgcctcaccc tagtcctggt ggcatttgtc tcaggtgcaa aatttaagaa agaaaccttg |
| 5581 | gagtgctcac cctgtggctg ggtagatggt cctaaagtgg tggttttcaa gcctgagtgt |
| 5641 | gtatcaggat catcagggga gcttgctaaa gagcagttcc tgcggtcaga ccctcatgca |
| 5701 | ttttgagcag gtgtggggac tgggaaactg catctgtaac ctgctgtaat ctaacgctta |
| 5761 | tctaaatact actgtgctca cacagagaac accgcaaaag tagaggtgtt cctccagagg |
| 5821 | gcaggtgagc agatggcaca gtctgcttgg aattcagtca ggtgatgaga gatgagatga |
| 5881 | ggcactccta gctttgggaa gagggagctg aaagatgaac ctttgcaggt gcccacggtc |
| 5941 | aaagtggtgg tttaatgcca tgccatgccc attttctgtt ggccttggca gggagttaca |
| 6001 | gccctacctt aggacctggc tccttatttc tgctgtaggc tctttcctgc cctggccgag |
| 6061 | atggagtgga atgagaccta gaaacatcaa gctaaataca tgtcctcaga aagataaagg |
| 6121 | tttacatttt cacccccatc aaatctgaaa gctctctgcc tgtgtttttc taagggatag |
| 6181 | ggacatcatt actcagtcca caacctggac tcatgtaggg tcccctgtca gtaaaggagt |
| 6241 | cagtcaagcc caccaggtat accaaggact cttaccctca gcccctactc cttggaaagc |
| 6301 | tgccccttgg cctaatattg gtgtttagct tgagcctgac tccttctcaa cactaagagc |
| 6361 | tgatgaagtc ctgaagcaga aagagctctg acctgagagt caaacatcct tattctgatc |
| 6421 | tcagctcagc ccctgatttg ttgtgtgacc ctggatatgt cacttcctgt ctttttgact |
| 6481 | ttttaaaatg aagggtagac tagaggagag cttctaaaac tttaatgtgg tcaacgaaat |
| 6541 | ggaataggaa attccacaag tctgtccttc cacaaaagca gcaaataagg tggcaaaaac |
| 6601 | tcaaatttat gggaactctg gaaacgaatt gaaagtttac agcaatcagg tgaataccta |
| 6661 | agaataaaag ctggatttag taaga |
| (SEQ ID NO: 17 or 75, 80, 85, 90, 95, 97, 98 or 100% identical thereto). | |
| 1 | msypgypppp ggyppaapgg gpwggaaypp ppsmppigld nvatyagqfn qdylsgmaan |
| 6 | msgtfgganm pnlypgapga gyppvppggf gqppsaqqpv ppygmypppg gnppsrmpsy |
| 121 | ppypgapvpg qpmpppgqqp pgaypgqppv typgqppvpl pgqqqpvpsy pgypgsgtvt |
| 181 | pavpptqfgs rgtitdapgf dplrdaevlr kamkgfgtde qaiidclgsr snkqrqqill |
| 241 | sfktaygkdl ikdlkselsg nfektilalm ktpvlfdiye ikeaikgvgt deaclieila |
| 301 | srsnehirel nraykaefkk tleeairsdt sghfqrllis lsqgnrdest nvdmslaqrd |
| 361 | aqelyaagen rlgtdeskfn avlsrsrah lvavfneyqr mtgrdieksi cremsgdlee |
| 421 | gmlavvkclk ntpaffaerl nkamrgagtk drtlirimvs rsetdlldir seykrmygks |
| 481 | lyhdisgdts gdyrkillki cggnd |

(SEQ ID NO: 18 or 75, 80, 85, 90, 95, 97, 98 or 100% identical thereto).

Annexin A13 is a protein that in humans is encoded by the ANXA13 gene (mRNA NM_004306; NM_001003954; protein NP_001003954; NP_004297).

| 1 | gcctgtagga ggactgatct cttgatgaaa tacagaaaaa ccatctcaga aaaaggaaaa |
|---|---|
| 61 | tgggcaatcg tcatgctaaa gcgagcagtc ctcagggttt tgatgtggat cgagatgcca |
| 121 | aaaagctgaa caaagcctgc aaaggaatgg ggaccaatga agcagccatc attgaaatct |
| 181 | tatcgggcag gacatcagat gagaggcaac aaatcaagca aaagtacaag gcaacgtacg |
| 241 | gcaaggagct ggaggaagta ctcaagagtg agctgagtgg aaacttcgag aagacagcgt |
| 301 | tggcccttct ggaccgtccc agcgagtacg ccgcccggca gctgcagaag gctatgaagg |
| 361 | gtctgggcac agatgagtcc gtcctcattg aggtcctgtg cacgaggacc aataaggaaa |
| 421 | tcatcgccat taaagaggcc taccaaaggc tatttgatag gagcctcgaa tcagatgtca |
| 481 | aaggtgatac aagtggaaac ctaaaaaaaa tcctggtgtc tctgctgcag gctaatcgca |
| 541 | atgaaggaga tgacgtggac aaagatctag ctggtcagga tgccaaagat ctgtatgatg |
| 601 | caggggaagg ccgctggggc actgatgagc ttgcgttcaa tgaagtcctg gccaagagga |
| 661 | gctacaagca gttacgagcc acctttcaag cctatcaaat tctcattggc aaagacatag |
| 721 | aagaagccat tgaagaagaa acatcaggcg acttgcagaa ggcctattta actctcgtga |
| 781 | gatgtgccca ggattgtgag gactattttg ctgaacgtct gtacaagtcg atgaagggtg |
| 841 | cggggaccga tgaggagacg ttgattcgca tagtcgtgac cagggccgag gtggaccttc |
| 901 | aggggatcaa agcaaagttc caagagaagt atcagaagtc tctctctgac atggttcgct |
| 961 | cagatacctc cggggacttc cggaaactgc tagtagccct cttgcactga gccaagccag |
| 1021 | ggcaatagga acacagggtg gaaccgcctt tgtcaagagc acattccaaa tcaaacttgc |
| 1081 | aaatgagact cccgcacgaa aacccttaag agtcccggat tactttcttg gcagcttaag |
| 1141 | tggcgcagcc aggccaagct gtgtaagtta agggcagtaa cgttaagatg cgtgggcagg |
| 1201 | gcaccttgaa ctctggctta gcaagcatct aggctgcctc ttcactttct tttagcatgg |
| 1261 | taactggatg ttttctaaac actaatgaaa tcagcagttg atgaaaaaac tatgcatttg |
| 1321 | taatggcaca tttagaagga tatgcatcac acaagtaagg tacaggaaag acaaaattaa |
| 1381 | acaatttatt aattttcctt ctgtgtgttc aatttgaaag cctcattgtt aattaaagtt |
| 1441 | gtggattatg cctcta |
| (SEQ ID NO: 19 or 75, 80, 85, 90, 95, 97, 98 or 100% identical thereto). | |
| 1 | mgnrhsqsyt lsegsqqlpk gdsqpstvvq plshpsrnge peapqpakas spqgfdvdrd |
| 61 | akklnkackg mgtneaaiie ilsgrtsder qqikqkykat ygkeleevlk selsgnfekt |
| 121 | alalldrpse yaarqlqkam kglgtdesvl ievlctrtnk eiiaikeayq rlfdrslesd |
| 181 | vkgdtsgnlk kilvsllqan rnegddvdkd lagqdakdly dagegrwgtd elafnevlak |
| 241 | rsykqlratf qayqiligkd ieeaieeets gdlqkayltl vrcaqdcedy faerlyksmk |
| 301 | gagtdeetli rivvtraevd lqgikakfqe kyqkslsdmv rsdtsgdfrk llvallh |

(SEQ ID NO: 20 or 75, 80, 85, 90, 95, 97, 98 or 100% identical thereto).

### Bacteria/External Stress

Bacteria in the method provided herein can be pathogenic and nonpathogenic bacteria, such as those that form filaments. These bacteria include gram-negative and gram-positive bacteria. Some examples include, but are not limited to, Acetobacter aurantius, Acinetobacter baumannii, Actinomyces israelii, Agrobacterium radiobacter, Agrobacterium tumefaciens, Anaplasma, Anaplasma phagocytophilum, Azorhizobium caulinodans, Azotobacter vinelandii, viridans streptococci, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides, Bacteroides fragilis, Bacteroides gingivalis, Bacteroides melaninogenicus (now known as Prevotella melaninogenica), Bartonella, Bartonella henselae, Bartonella quintana, Bordetella, Bordetella bronchiseptica, Bordetella pertussis, Borrelia burgdorferi, Brucella, Brucella abortus, Brucella melitensis, Brucella suis, Burkholderia, Burkholderia mallei, Burkholderia pseudomallei, Burkholderia cepacian, Calymmatobacterium granulomatis, Campylobacter, Campylobacter coli, Campylobacter fetus, Campylobacter jejuni,

Campylobacter pylori, Capnocytophaga canimorsus, Chlamydia, Chlamydia trachomatis, Chlamydophila, Chlamydophila pneumoniae (previously called Chlamydia pneumoniae), Chlamydophila psittaci (previously called Chlamydia psittaci), Citrobacter, Citrobacter freundii,
Citrobacter koseri, Clostridium, Clostridium botulinum, Clostridium difficile, Clostridium perfringens (previously called Clostridium welchii), Clostridium tetani, Corynebacterium, Corynebacterium diphtheriae, Corynebacterium fusiforme, Coxiella burnetiid, Cutibacterium acnes (previously called Propionibacterium acnes), Ehrlichia chaffeensis, Ehrlichia ewingii, Eikenella corrodens, Enterobacter cloacae, Enterococcus, Enterococcus avium, Enterococcus durans, Enterococcus faecalis, Enterococcus faecium, Enterococcus gallinarum, Enterococcus maloratus, Escherichia coli, Francisella tularensis (previously called Pasteurella tularensis), Fusobacterium necrophorum, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Kingella kingae, Klebsiella, Klebsiella granulomatis, Klebsiella pneumoniae, Lactobacillus, Lactobacillus acidophilus, Lactobacillus bulgaricus, Lactobacillus casei, Lactococcus lactis, Legionella pneumophila, Leptospira interrogans, Leptospira noguchii, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans,
Mycoplasma genitalium, Mycoplasma hominis, Mycoplasma penetrans, Mycoplasma pneumoniae, Mycoplasma Mexican, Neisseria, Neisseria gonorrhoeae, Neisseria meningitidis, Nocardia, Nocardia asteroids, Nocardia brasiliensis, Nocardia cyriacigeorgica, Nocardia farcinica, Pasteurella multocida, Peptostreptococcus, Porphyromonas gingivalis, Prevotella melaninogenica (previously called Bacteroides melaninogenicus), Proteus, Proteus mirabilis, Proteus penneri, Proteus vulgaris, Pseudomonas aeruginosa, Rhizobium radiobacter, Rickettsia, Rickettsia prowazekii, Rickettsia psittaci, Rickettsia quintana, Rickettsia rickettsia, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Spirillum volutans, Staphylococcus, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Ureaplasma urealyticum, Vibrio, Vibrio cholerae, Vibrio comma, Vibrio parahaemolyticus, Vibrio vulnificus, Wolbachia, Yersinia, Yersinia enterocolitica, Yersinia pestis, and/or Yersinia pseudotuberculosis.

### External Stress

External stress (a stressor that can induce a stress response in bacteria can be any condition outside of the ideal conditions for survival) can include, but is not limited to, antibiotics, malnutrition (e.g., nutrient deprivation), chemical exposure (e.g., reactive oxygen or chlorine species), exposure to oxidants, hypo/hyper-osmolarity, pH changes (e.g., extreme pHs), temperature changes (such as sudden increases or decreases in temperature), heat, and/or cold, and can also lead to the formation of filamentous bacteria (bacteria with longer filaments than similar bacteria not undergoing stress).

Antibiotics can include, but are not limited to:

ß-Lactam antibiotics are a class of antibiotics that includes penicillins, cephalosporins, monobactams, and carbapenems, all of which contain a β-lactam ring.

Antibiotics of the penicillin class are bactericidal and work by inhibiting the synthesis of bacterial cell walls, they include but are not limited to: Amoxicillin, Ampicillin, Bacampicillin, Carbenicillin, Cloxacillin, Dicloxacillin. Flucloxacillin, Mezlocillin, Nafcillin, Oxacillin, Penicillin G, Penicillin V, Piperacillin, Pivampicillin, Pivmecillinam and Ticarcillin.

Cephalosporins are bactericidal and act by inhibiting the synthesis of the peptidoglycan layer of bacterial cell walls. Besides true cephalosporins (which are derived from cephalosporin C) this class includes oxacephems and carbacephems. Cephalosporins include, but are not limited to, Cefacetrile (cephacetrile), Cefadroxil (cefadroxyl), Cefalexin (cephalexin), Cefaloglycin (cephaloglycin), Cefalonium (cephalonium), Cefaloridine (cephaloradine), Cefalotin (cephalothin), Cefapirin (cephapirin), Cefatrizine, Cefazaflur, Cefazedone, Cefazolin (cephazolin), Cefradine (cephradine), Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, and Ceftioxide.

Monobactams, including, but not limited to, aztreonam.

Carbapenems, including, but not limited to, Imipenem, Imipenem/cilastatin, Doripenem, Ertapenem, Meropenem, and Meropenem/vaborbactam.

Macrolide antibiotics, including, but not limited to, Azithromycin, Erythromycin, Clarithromycin, Dirithromycin, Roxithromycin, and Ketolides, such as Telithromycin.

Lincosamides, including, but not limited to, Clindamycin, and Lincomycin.

Streptogramins, including, but not limited to, Pristinamycin and Quinupristin/dalfopristin.

Aminoglycoside Antibiotics, including, but not limited to, Amikacin, Gentamicin, Kanamycin, Neomycin, Netilmicin, Paromomycin, Streptomycin, and Tobramycin.

Quinolone Antibiotics, including, but not limited to, Flumequine, Nalidixic acid, Oxolinic acid, Piromidic acid, Pipemidic acid, Rosoxacin, Ciprofloxacin, Enoxacin, Lomefloxacin, Nadifloxacin, Norfloxacin, Ofloxacin, Pefloxacin, Rufloxacin, Balofloxacin, Gatifloxacin, Grepafloxacin, Levofloxacin, Moxifloxacin, Pazufloxacin, Sparfloxacin, Temafloxacin, Tosufloxacin, Besifloxacin, Delafloxacin, Clinafloxacin, Gemifloxacin, Prulifloxacin, Sitafloxacin, and Trovafloxacin.

Sulfonamides, including, but not limited to, Sulfamethizole, Sulfamethoxazole, Sulfisoxazole, and Trimethoprim-Sulfamethoxazole.

Tetracycline Antibiotics, including, but not limited to, Demeclocycline, Doxycycline, Minocycline, Oxytetracycline, Tetracycline and Glycylcyclines, such as Tigecycline.

Others include, but are not limited to, Chloramphenicol, Metronidazole, Tinidazole, Nitrofurantoin, Glycopeptides, Vancomycin, Teicoplanin, Lipoglycopeptides, Telavancin, Oxazolidinones, Linezolid, Cycloserine, Rifamycins, Rifampin, Rifabutin, Rifapentine, Rifalazil, Polypeptides, Bacitracin, Polymyxin B, Tuberactinomycins, Viomycin and Capreomycin.

### Expression Vectors/Phage

To prepare expression cassettes encoding annexin, a peptide thereof, or a fusion thereof, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target. In one embodiment, expression is inducible.

Other elements functional in the host cells, such as introns, enhancers, polyadenylation sequences and the like, may also be a part of the recombinant DNA. Such elements may or may not be necessary for the function of the DNA but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

An annexin protein for use herein can be produced in a number of ways. In one embodiment, a coding sequence for annexin can be placed in a transfer or movable vector, such as a plasmid, and the plasmid is cloned into an expression vector or expression system. The expression vector for producing annexin may be suitable for E. coli, Bacillus, or a number of other suitable bacteria. The vector system may also be a cell free expression system. All of these methods of expressing a gene or set of genes are known in the art.

Bacteriophage vectors are known in the art an include filamentous phages (e.g., M13), bacteriophage lambda and bacteriophage P1. Annexin can be cloned into a bacteriophage vector.

Provided are compositions containing nucleic acid molecules that, either alone or in combination with other nucleic acid molecules, are capable of expressing an effective amount of annexin in vivo. Cell cultures containing these nucleic acid molecules, polynucleotides, and vectors carrying and expressing these molecules in vitro or in vivo, are also provided.

### Peptides, Polypeptides and Fusion Proteins

The peptide or fusion proteins of the invention can be synthesized in vitro, e.g., by the solid phase peptide synthetic method or by recombinant DNA approaches (see above). The solid phase peptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, chemically modified derivatives of a given peptide or fusion thereof, can be readily prepared. For example, amides of the peptide or fusion thereof of the present invention may also be prepared by techniques well known in the art for converting a carboxylic acid group or precursor, to an amide. One method for amide formation at the C-terminal carboxyl group is to cleave the peptide or fusion thereof from a solid support with an appropriate amine, or to cleave in the presence of an alcohol, yielding an ester, followed by aminolysis with the desired amine.

Salts of carboxyl groups of a peptide or fusion thereof may be prepared in the usual manner by contacting the peptide, polypeptide, or fusion thereof with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide or fusion thereof may be prepared by utilizing an N-acyl protected amino acid for the final condensation, or by acylating a protected or unprotected peptide, polypeptide, or fusion thereof. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like. Both N-and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

Substitutions may include substitutions which utilize the D rather than L form, as well as other well-known amino acid analogs, e.g., unnatural amino acids such as α,α-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, omithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, ω-N-methylarginine, and other similar amino acids and imino acids and tert-butylglycine.

Conservative amino acid substitutions may be employed-that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the invention, are, in general, accomplished by selecting substitutions that do not differ significantly in their effect on maintaining (a) the structure of the peptide backbone in the area of the substitution. (b) the charge or hydrophobicity of the molecule at the target site, or (c) the bulk of the side chain. Naturally occurring residues are divided into groups based on common side-chain properties:
(1) hydrophobic: norleucine, met, ala, val, leu, lie;
(2) neutral hydrophilic: cys, ser, thr;
(3) acidic: asp, glu;
(4) basic: asn, gin, his, lys, arg;
(5) residues that influence chain orientation: gly, pro; and
(8) aromatic; trp, tyr, phe.

The invention also envisions a peptide, polypeptide or fusion polypeptide with non-conservative substitutions. Non-conservative substitutions entail exchanging a member of one of the classes described above for another.

Acid addition salts of the peptide, polypeptide or fusion polypeptide or of amino residues of the peptide, polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

### Formulations/Dosages

Therapeutic or pharmaceutical compositions comprising annexin for use in the methods and applications are provided herein. Therapeutic or pharmaceutical compositions may comprise annexin (or an expression vector coding for annexin, such as a plasmid or phage), optionally combined with one or more antibiotics, optionally combined with suitable excipients, carriers or vehicles. Further, annexin can optionally be included in the carrier. The invention provides therapeutic compositions or pharmaceutical compositions of annexin in combination with at least one antibiotic for use in the killing, alleviation, decolonization, prophylaxis or treatment of bacteria, including bacterial infections or related conditions. Annexin can be provided as a peptide, as an RNA (similar to COVID vaccines), or as part of a vector, such as a plasmid or bacteriophage.

The polypeptides or nucleic acid encoding the polypeptides of the invention (including vectors), can be formulated as pharmaceutical compositions and administered to a mammalian host, such as a human patient in a variety of forms adapted to the chosen route of administration, e.g., orally or parenterally, by intravenous, intramuscular, topical or subcutaneous routes. In one embodiment, the polypeptide or nucleic acid encoding the polypeptide is administered to a site of cartilage damage or suspected cartilage damage or is administered prophylactically.

In one embodiment, annexin or nucleic acid encoding the polypeptide may be administered by infusion or injection. Solutions of the polypeptides thereof, or nucleic acid encoding the polypeptide or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze-drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the polypeptides or nucleic acid encoding the polypeptide can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the polypeptides or nucleic acid encoding the polypeptide in a liquid composition, may be from about 0.1-25 wt-%, e.g., from about 0.5-10 wt-%. The concentration in a semi-solid or solid composition such as a gel or a powder may be about 0.1-5 wt-%. e.g., about 0.5-2.5 wt-%.

The amount of the polypeptides or nucleic acid encoding the polypeptide required for use alone or with other agents will vary with the route of administration, the nature of the condition being treated and the age and condition of the patient and will be ultimately at the discretion of the attendant physician or clinician.

The polypeptides or nucleic acid encoding the polypeptide may be conveniently administered in unit dosage form; for example, containing 5 to 1000 mg, conveniently 10 to 750 mg, or conveniently 50 to 500 mg of active ingredient per unit dosage form.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

Further, the effective dosage rates or amounts of annexin for use in the present invention will depend in part on whether the annexin will be used therapeutically or prophylactically, the duration of exposure of the recipient to the infectious bacteria, the size and weight of the individual, etc. The duration for use of the composition containing the annexin polypeptide also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. Carriers that are classified as “long” or “slow” release carriers (such as, for example, certain nasal sprays or lozenges) could possess or provide a lower concentration of active (protein) units per ml, but over a longer period of time, whereas a “short” or “fast” release carrier (such as, for example, a gargle) could possess or provide a high concentration of active (protein) units per ml, but over a shorter period of time. The amount of active units per ml and the duration of time of exposure depend on the nature of infection, whether treatment is to be prophylactic or therapeutic, and other variables. There are situations where it may be necessary to have a much higher unit/ml dosage or a lower unit/ml dosage.

The annexin and antibiotic(s) may be administered simultaneously or subsequently. The annexin and antibiotic agent(s) may be administered in a single dose or multiple doses, singly or in combination. The annexin and antibiotic may be administered by the same mode of administration or by different modes of administration, and may be administered once, twice or multiple times, one or more in combination or individually. Thus, annexin may be administered in an initial dose followed by a subsequent dose or doses, particularly depending on the response and bacterial killing or decolonization and may be combined or alternated with antibiotic dose(s). In a particular aspect of the invention and in view of the reduction in the development of resistance to antibiotics by administering annexin with antibiotic, combinations of antibiotic and annexin may be administered for longer periods and dosing can be extended without risk of resistance. In addition, in as much as the doses required for efficacy of each of antibiotic and annexin are significantly reduced by combining or co-administering the agents simultaneously or in series, a patient can be treated more aggressively and more continually without risk, or with reduced risk, of resistance.

The following Example illustrates some of the materials, methods, and experiments that were used or performed in the development of the invention.

### EXAMPLE

### Introduction

E. coli and many other bacteria under conditions of stress reduce the frequency of undergoing cytokinesis while continuing to grow and replicate their genome. The result is transformation of the bacteria into a filamentous form[1]. For infectious bacteria this may have survival value by preventing consumption of the bacteria by phagocytic cells of the immune system of the host organism, and the formation of biofilms that protect bacterial colonies. Provided herein is the unexpected discovery that the expression of mammalian annexin A4, a calcium-dependent, phospholipid-binding protein, in E. coli restores the ability for bacteria undergoing antibiotic stress to undergo cytokinesis and leads to a return to a normal morphology.

The annexins constitute a highly conserved family of calcium-binding proteins in plants, animals, and protists [2,3]. However, they are not expressed in E. coli. Motifs similar to the canonical fourfold calcium-and membrane-binding motifs in eukaryotic annexins [4] have been discovered in proteins in some strains of bacteria [5,6]. Their occurrence in bacteria is rare, however. The bioinformatic analysis of Kodavali et al [6] detected annexin motifs in only 17 bacterial species among the thousands of genomes analyzed. It is unclear if the bacterial annexin-like proteins evolved independently from eukaryotic annexins or if genetic material encoding annexin motifs was acquired by non-genetic transfer from the eukaryotic relatives. The bacterial annexin-like proteins are unusual in that they do not generally follow the pattern of the fourfold domain repeat found in the eukaryotic examples as they have as few as a single annexin motif and up to 6 copies of the repeats. None of the bacterial proteins containing annexin motifs have yet been isolated or characterized biochemically so it is not known if they bind calcium and membranes, although the sequence similarities to the eukaryotic proteins suggest this may be the case. Nor is there evidence for any particular function of the bacterial annexins obtained, for example, by gene deletion or modification experiments. Therefore, the ability of the eukaryotic annexin to modulate a specific function in bacteria, cytokinesis, was not anticipated.

The affinity of eukaryotic annexins for calcium is modulated by the coincident binding of phospholipids [7,8] so the annexins are implicated in many activities regulated by calcium that occur with or on membranes. It has been reported that inhibition of the expression of a particular eukaryotic annexin, annexin A11, in human A431 and HeLa cells leads to the failure of cytokinesis in its final stage during which the neck of cytoplasm between daughter cells undergoing mitosis collapses and the membranes fuse, separating the cells [9]. It is not known what the precise role for the annexin may be in this process, but regulation by calcium, and the ability to pull two membranes together to promote membrane fusion could provide an explanation consistent with the known in vitro properties of the annexins [10].

In 1940 Gardner reported that, while working with Chain, Florey, and others at Oxford, he observed that many types of bacteria exposed to penicillin at low concentrations formed long filaments [11,12]. Subsequently it was determined that this morphological change was caused by the binding of penicillin or other beta-lactam antibiotics to proteins that play roles in bacterial cell division [13-15]. For example, in E. coli penicillin binds to penicillin binding protein 3 (PB3) which functions in peptidoglycan synthesis in the bacterial cell wall, including in the septa between dividing cells. This process is inhibited by the binding of penicillin resulting in continued growth of the bacteria without cell division, producing filaments containing multiple copies of the bacterial genome.

It has been suggested that the formation of these filaments may provide a selective advantage to bacteria under attack by phagocytic cells of the immune system of eukaryotic organisms which are unable to encapsulate the extended bacterial filaments [1,16]. To this extent the formation of bacterial filaments may be of medical importance in that they may impair treatment of bacterial infections with beta-lactam antibiotics. The result is that bacteria gain resistance to the host defenses precisely when the antibiotic is administered with the intention to inhibit bacterial growth. It is possible that small molecule drugs that could inhibit the filamentation process might provide an effective adjuvant treatment to enhance antibiotic efficacy, but no such compounds have been identified. The results reported herein demonstrate that expression of annexins in pathogenic bacteria can serve such a prophylactic benefit.

### Materials And Methods

### Materials

Components of the bacterial growth medium, Luria broth, (10 gm/liter tryptone, 5 gm/liter NaCl, yeast extract 10 gm/liter) were obtained from Fisher (Fair Lawn, New Jersey). Bacterial agar for petri plates was from Acros Organics (The Hague, Netherlands), ampicillin from Fisher (BP1760), piperacillin from Sigma-Aldrich (P8396) (St. Louis, Missouri), cephalexin from Alpha Aesor (J63172) (Haverhill, Massachusetts), IPTG from Fisher, and glutaraldehyde from Fisher. Stock concentrations of the antibiotics were prepared as follows and stored at minus 20 degrees C.: ampicillin 100 mg/ml in 1:1 ethanol:H₂O; piperacillin 2 mg/ml in ethanol; and cephalexin 25 mg/ml in 1:1 ethanol:H₂O. Other standard chemicals were of reagent grade obtained from Fisher or Sigma.

E. coli strain BL21(DE3) was obtained from Thermofisher Scientific (Waltham, Massachusetts) and the expression plasmid pET11d from Millipore Sigma (Burlington, Massachusetts).

Annexin expression plasmids were constructed as described [17,18]. Since the plasmids were originally sequenced manually in the early 1990's the inserts in all plasmids used in this study were resequenced with high fidelity automated Sanger sequencing by Azenta Life Sciences, (Burlington, Massachusetts). All designed mutations were confirmed, and no spurious mutations were found.

### Methods

Use of dark field light microscopy to monitor antibiotic-driven bacterial filament formation.

In bright field light microscopy, the filaments formed by bacteria in the presence of beta-lactam antibiotics are difficult to distinguish from “floaters” in the eyes of the microscopist. This problem is eliminated by using dark field microscopy because the visual floaters do not scatter light from the microscope condenser into the objective lens and are therefore invisible. The visual floaters also do not appear in micrographs taken by photography or videography in either brightfield or darkfield. The light micrographs provided herein were all obtained by darkfield light microscopy. This technique also enhanced the contrast of the bacteria against a dark background and led to easy visualization of areas of difference of refractive index such as the difference between the cell wall and cytoplasm. This was particularly useful for monitoring overall shape and real-time growth of bacterial cells; however, intracellular structures were generally not resolved. Therefore, transmission electron microscopy was also used in some cases for visualization of cell ultrastructure.

Darkfield light microscopy of bacterial cultures was performed with a Nikon Labophot microscope (Nikon Instruments, Melville, New York) with a dark field condenser (dry) and an e plan40×/0.65 Nikon objective. Micrographs were recorded with an Amscope Microscope digital camera MU 1803-HS 18mp Aptina color CMOS (Irvine, California) using software supplied with the camera.

### Cell Culture and Microscopy

Protein expression from the plasmid pET11d can be regulated by the addition

of the inducer IPTG (Isopropyl β-d-1-thiogalactopyranoside) [19]. However, protein expression from this plasmid can have significant baseline expression without the inducer. It was seen in SDS gels of extracts of bacteria expressing annexin A4 that basal expression of the annexin appeared to be 5 to 10% of expression seen with the inducer [20]. In preliminary tests for the present study it was found that including the inducer (concentration 0.4 mM, previously used for maximum protein production) led to formation of inclusion bodies of insoluble protein, distortion of bacterial morphology and partial lysis and aggregation of the bacteria. Accordingly, all of the imaging and experiments described in this report were performed without adding the inducer, which avoided these problems.

Five ml precultures in LB medium with 200 μg/ml ampicillin were seeded with colonies from LB-ampicillin agar plates and incubated on a rotator overnight at 34 degrees C. Fifty μl samples of the saturated cultures were diluted into 5 ml of LB medium with the specified antibiotics in 17×100 mm clear polystyrene culture tubes with loose fitting caps that permitted gas exchange (Fisher Healthcare 14-956-6B) and the growth of the cultures at 34 degrees C. was monitored by dark field microscopy and measurement of absorbance at a wavelength of 600 nm. For the microscopy, 20 ul samples of the culture were placed on a glass slide (Fisher, 12-550123 1×3 inches, 1 mm thickness, precleaned by the manufacturer) and the drop of culture fluid covered with a cover slip (Fisher 12540B 22×22 mm, 0.17-0.25 mm thickness). Slides were rested for 2 to 5 minutes to allow the cells to settle before observation. Nonetheless, the cells were not tightly adherent to the slides and occasionally some cells moved slightly during imaging resulting in a double image. Cultures were checked and micrographs taken at 5 to 20 min intervals for up to 4 hours. Occasionally during this period, the culture tubes were briefly inserted into a Genesys 20 spectrophotometer (Thermospectronic, Rochester, NY) and the absorbance at 600 nm was recorded to measure culture density. The culture tubes have a circular cross section with an inside diameter of 1.3 cm, so the absorbances recorded were approximately 30% higher than would have been measured in a square cuvette with a one cm pathlength.

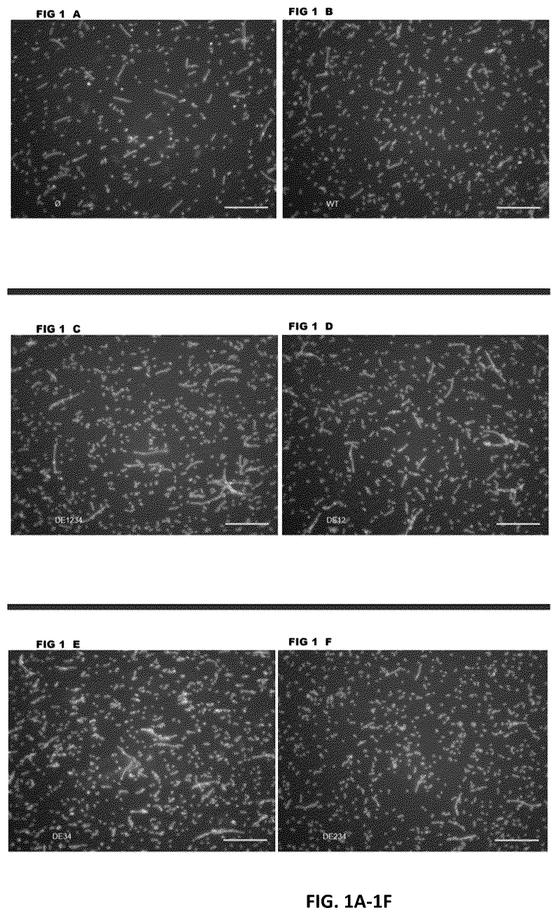

For preparation of the micrographs in FIGS. 1 through 3 comparing the morphology of the different transformed strains, each test culture (5 ml) was inoculated at the same time with 50 ul of the saturated overnight preculture. For display in the Figures, photographs taken after approximately the same period of growth of each of the parallel cultures were obtained when 20 μl samples provided a microscope image containing a broad sampling of the cell morphologies seen without overcrowding. Each experiment illustrated was repeated at least three times with comparable results.

### Measurement and Statistical Analysis of Filament Lengths

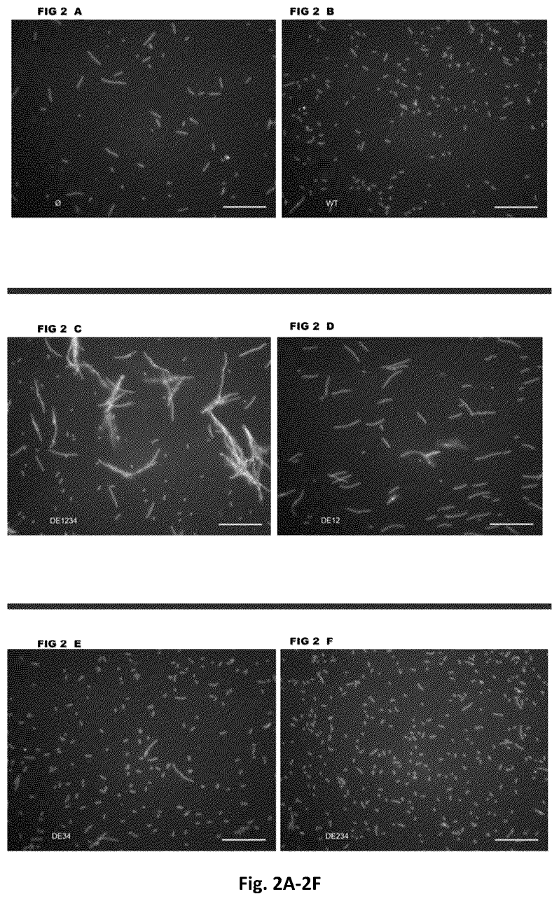

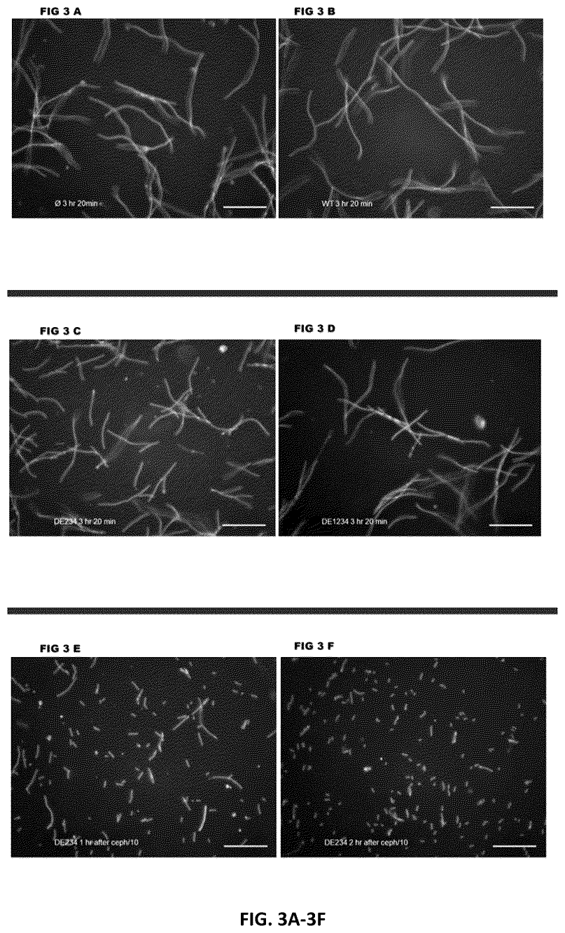

The lengths of the bacterial filaments were measured using the Image J program (NIH) with the dark field light micrographs. Particles that did not have any linear dimension greater than 2 μm were not measured because of uncertainty as to whether they represented bacterial cells, broken cells, or other debris in the light micrographs. The longest dimension of each cell or filament was taken to represent the “length” for the analysis. Forty to 90 filaments were measured from each of the sub-images in FIGS. 1 and 2. In FIG. 3 the lengths were not determined because most filaments extended beyond the dimensions of the images and were greater than 200 um. It was instead recorded if these filaments were stable, or broke up into shorter filaments over time, as seen in some of the panels in FIG. 3.

### Electron Microscopy

E. coli samples selected for ultrastructural analysis were processed for electron microscopy using technical support and instrumentation in the Advanced Microscopy Facility at the University of Virginia School of Medicine. The following reagents for electron microscopy were obtained from Electron Microscopy Sciences (Hatfield, PA): sodium cacodylate, propylene oxide, EPON, uranyl acetate, and lead citrate.

One ml samples of cell cultures were pelleted in a microcentrifuge and resuspended in 2.5% glutaraldehyde in 10 mM phosphate buffer (pH7.4) and held overnight at 4 degrees C. The cells were then washed in 0.1 M sodium cacodylate, 2 times, 10 minutes per wash. Then the samples were completely immersed in 1% osmium tetroxide (OsO4) in 0.1 M sodium cacodylate and incubated at room temperature for 1 hour. Following this incubation, the samples were rinsed in 0.1 M sodium cacodylate, 2 times, 10 minutes per wash. Next samples were dehydrated sequentially in 50%, 70%, 95%, 100% ethanol for at least 10 minutes each. Samples were then rinsed in 1:1 ethanol:propylene oxide and incubated for 10 min in the same mixture, then for 10 min in 100% propylene oxide. Following removal of the final propylene oxide, samples were incubated in a 1:1 propylene oxide:EPON mixture overnight, followed with 1:2 propylene oxide:EPON and 1:4 propylene oxide:EPON, 4 hours each. Following the initial fixation in glutaraldehyde all steps were done at room temperature. Finally, samples were placed in an oven and incubated in 100% EPON overnight at 60 degrees C.

Following polymerization of the embedding medium, a clean razor blade was used to cut out the region of interest of the sample. After trimming the block to contain only the desired region, a Leica EM UC7 Ultramicrotome was used to cut ultrathin sections at 80 nm. Sections were collected on 200 mesh copper grids and counterstained using uranyl acetate and lead citrate. The grids were coated with a thin layer of carbon using a Leica ACE600 coating system to minimize conductance and improve stability in the electron microscope. Samples were stored at room temperature until imaging. Ultrathin sections were visualized with a JEOL JEM1230 transmission electron microscope.

### Results

### Morphology of E. coli Expressing Wild Type and Mutant Annexins A4 (the “DE” Mutants)

In 1993 Michael Nelson described the construction of a combinatorial library of mutant annexin A4 cDNAs that was designed to facilitate study of the roles of each of the four homologous calcium and membrane binding sites in bovine annexin A4 in biochemical studies [18][17]. The annexin cDNAs in the library were expressed in E. coli using the prokaryotic expression vector pET11d. Each calcium binding site contains an important acidic amino acid residue, aspartate or glutamate, that contributes to the coordination of calcium in the cation binding site. Replacement of these residues with alanine by site directed mutation was used to create a library of mutant proteins with reduced calcium and membrane binding properties, which correlated with the specific combinations of mutations present. These mutants were called the “DE” mutants, for replacement of aspartate (D) or glutamate (E). The sites of the mutations are denoted by the domains harboring the mutations. For example, the DEI mutant has the calcium binding site in the first domain mutated. The DE12 mutant has the calcium binding sites in the first and second domains mutated, and so forth up to the DE1234 mutant which has all four sites mutated. The specific residues mutated in the constructs used in the present study are listed in the paper by Nelson and Creutz [17]. These mutations reduced the calcium sensitivity of the annexin by up to an order of magnitude in two types of assays: binding of the annexin to a biological membrane (chromaffin granule membrane) and aggregation of chromaffin granule membranes. The calcium sensitivities were altered depending upon which mutations, or combination of mutations were introduced (see Nelson and Creutz [17], Table 4). The concentration of calcium leading to half-maximal binding to a biological membrane (bovine chromaffin granule (secretory vesicle) membrane were as follows: wild type, 5.0×10⁻⁶ M; DE12, 4.0×10⁻⁵ M; DE34, 4.9×10⁻⁵ M; DE234, 1.1×10⁻⁴ M; DE1234—no binding. However, it should be noted that the calcium binding affinities of annexins depend on a lipid headgroup participating as a ligand for calcium in the calcium binding pocket. Therefore, the exact affinity for calcium depends on the specific lipid headgroups participating in the binding. Although the annexins generally bind lipids with negatively charged headgroups, the specific lipids in E. coli that are bound by the annexin have not been determined. Therefore, the binding affinities in E. coli are not known precisely but are likely to be similar. SDS gel analysis of bacterial extracts indicated that all of the mutants in the library were expressed in the bacteria to comparable levels (Nelson and Creutz (17)).

As demonstrated in the sections below, the use of selected mutants from this library revealed that the ability of the annexin to promote cytokinesis in bacteria was dependent on the competence of some of these sites. This suggests that the restoration of the process of cytokinesis in antibiotic-treated bacteria by annexin expression is calcium-dependent.

Control cultures grown in ampicillin (200 μg/ml) without expression of the annexin (FIG. 1A).

Dark field light microscopy of cultures of E. coli strain BL21DE3 harboring the expression plasmid pET11d without an annexin cDNA insert grown in LB-ampicillin media reveals a broad distribution of bacterial cell lengths from 2 to 25 μm (average 9.645 um, SD (sample standard deviation) 0 5.449 um, n=81).

Bacterial cultures grown in ampicillin expressing wild type annexin A4 (FIG. 1B).

When the bacteria were transformed with the expression plasmid containing the wild type annexin A4 cDNA, the number of longer, filamentous forms of the bacteria were reduced relative to the shorter forms (average 5.279 um, SD 2.535 um, n=62), although the optical density (A600) of the cultures with or without the annexin were similar, suggesting net growth of bacterial mass was not greatly altered.

Bacterial cultures grown in ampicillin expressing the DE1234 mutant annexin A4 (FIG. 1C).

When bacteria were transformed with the expression plasmid containing the DE1234 mutant annexin, with all four calcium binding sites mutated, a number of longer filaments were formed that were up to 37 μm long (average 8.486 um, SD 6.580 um, n=61)—similar to the ones that formed when the bacteria were transformed with the empty expression vector (FIG. 1A). It therefore appeared that the annexin in which all the calcium binding sites had been had a similar effect to removing the entire annexin cDNA from the vector.

Bacterial cultures grown in ampicillin expressing the DE12 mutant annexin A4 (FIG. 1D).

When bacteria were transformed with a plasmid expressing the DE12 mutant, which is defective in the calcium binding sites in domains 1 and 2 but has normal calcium binding sites in domains 3 and 4, the cultures had a distribution of sizes (average 9.139 um, SD 4.104 um, n=65) similar to control cells transformed with the empty plasmid (FIG. 1A). This suggests that domains 1 and 2 play a dominant role in mediating the cytokinesis promoting effect, while domains 3 and 4 play a minor role in promoting this effect.

Bacterial cultures grown in ampicillin expressing the DE34 mutant annexin A4 (FIG. 1E).

When the bacteria were transformed with the plasmid expressing the DE34 mutant, which has the calcium binding sites in domains 3 and 4 mutated but retains wild type calcium binding sites in domains 1 and 2, there were fewer long filaments and short 1 to 5 μm bacteria were more prominent (average 5.038 μm, SD 2.938 μm, n=61). The culture therefore resembled cultures transformed with the plasmid expressing the annexin with wild type calcium binding sites in all four domains (FIG. 1B). The calcium binding sites in the first two domains of the annexins are the most highly conserved in sequence and the results suggest these two sites provide the major contribution to the cytokinesis promoting activities of the unmutated protein.

Bacterial cultures grown in ampicillin expressing the DE234 mutant annexin A4 (FIG. 1F).

When bacteria were transformed with the construct expressing the DE234 mutant the culture looked most similar to cultures expressing the wild type annexin (average 4.643 μm, SD 2.577 μm, n=60). This result is notable in that this mutant only has one functional calcium-binding domain—domain 1—so this single domain appears to be sufficient to support the cytokinesis promoting activity of the intact annexin.

Statistical summary of filament lengths of cells grown in ampicillin (200 μg/ml).

Summary statistics for filaments grown in ampicillin are given in Table 1. P values for the differences between filament lengths of cells expressing different annexin constructs are given in Table 2. The behavior of the constructs fell into two distinct groups: Constructs that promoted cytokinesis (wild type annexin, DE34, and DE234) and constructs that did not promote cytokinesis (Ø (the null vector), DE12, and DE1234). The first group all expressed an annexin with a native (unmutated) first domain calcium binding site. The second group all had a mutated or absent first domain calcium binding site. The P values for comparisons of members of the first group with members of the second group were all highly significant: P values of 0.0005 or less.

TABLE 1
Filament lengths in FIG. 1 (ampicillin).

| Micrograph | Construct | n | AVG μm | SD μm | MAX μm |
|---|---|---|---|---|---|
| FIG. 1A | Ø | 81 | 9.645 | 5.449 | 24.88 |
| FIG. 1B | wt | 62 | 5.279 | 2.535 | 15.03 |
| FIG. 1C | DE1234 | 61 | 8.486 | 6.580 | 37.41 |
| FIG. 1D | DE12 | 65 | 9.139 | 4.104 | 23.49 |
| FIG. 1E | DE34 | 61 | 5.038 | 2.938 | 19.32 |
| FIG. 1F | DE234 | 60 | 4.643 | 2.577 | 15.26 |

Micrograph: Individual images in FIG. 1. Construct: Annexin expression construct: Ø, null vector with no annexin insert; wt, wildtype annexin A4; DE1234, etc -specific DE mutants with the mutated domains (1 through 4) indicated. n: Number of filaments measured in the designated micrograph. AVG μm: average length of the measured filaments; SD μm: sample standard deviation of the lengths of filaments; MAX μm: length of the longest filament in the micrograph.

TABLE 2
Statistical analysis of the differences in filament lengths in Table 1 (ampicillin).

| | | | | |
|---|---|---|---|---|
| Ø − wt 9.645, 5.279 | | | | |
| P < 0.0001 | | | | |
| Ø − DE1234 | wt − DE1234 | | | |
| 9.645, 8.486 | 5.279, 8.486 | | | |
| P = 0.2536 | P = 0.0005 | | | |
| Ø − DE12 | wt − DE12 | DE1234 − DE12 | | |
| 9.645, 9.139 | 5.279, 9.139 | 8.486, 9.139 | | |
| P = 0.5359 | P < 0.0001 | P = 0.5023 | | |
| Ø − DE34 | wt − DE34 | DE1234 − DE34 | DE12 − DE34 | |
| 9.645, 5.038 | 5.279, 5.038 | 8.486, 5.038 | 9.139, 5.038 | |
| P < 0.0001 | P = 0.6269 | P = 0.0003 | P < 0.0001 | |
| Ø − DE234 | wt − DE234 | DE1234 − DE234 | DE12 − DE234 | DE34 − DE234 |
| 9.645, 4.643 | 5.279, 4.643 | 8.486, 4.643 | 9.139, 4.643 | 5.038, 4.643 |
| P < 0.0001 | P = 0.1720 | P < 0.0001 | P < 0.0001 | P = 0.4336 |

Each entry in the table is organized as is the data in the top left entry: Ø − wt compares the lengths of filaments harboring the null vector (Ø, no annexin) and filaments expressing the wild-type annexin (wt); 9.645, 5.279 compares the lengths of the two types of filaments; P < 0.0001, P value for the significance of the difference in lengths. Calculations made from data in Table 1.

Studies with ampicillin (200 μg/ml) plus piperacillin (4 μg/ml) (FIG. 2)

Ampicillin binds penicillin binding protein 3 (PB3)[15]. A related beta-lactam antibiotic, piperacillin, binds PB3 with higher specificity for PB3 than other beta-lactam antibiotics and at low concentrations affects the formation of septa between dividing bacterial cells to a greater extent than elongation of cells [21]. Piperacillin was tested in this study as it was thought it might be more effective at promoting filament formation by the bacteria and therefore provide a better model for detecting the effects of the annexin on cytokinesis. As described in the sections below, differences between the DE mutants in affecting cytokinesis were much more apparent in cultures treated with ampicillin plus piperacillin.

Control bacterial cultures in ampicillin plus piperacillin without expression of annexin A4 (FIG. 2A).

As shown in FIG. 2A, bacteria harboring the empty expression plasmid and grown in 200 μg/ml ampicillin supplemented with 4 μg/ml piperacillin predominantly formed intermediate length filaments 10 to 40 μm long, with very few bacteria of near normal 2 to 4 μm length (average 10.71 μm, SD 5.520 μm, n=43).

Bacterial cultures in ampicillin and piperacillin expressing wild type annexin A4 (FIG. 2B).

When the bacteria were transformed with the plasmid expressing the wild type annexin A4 (FIG. 2B) the filament lengths were predominantly reduced to a length of 2 to 10 μm (average 5.052 μm, SD 1.594 μm, n=81).

Bacterial cultures in ampicillin and piperacillin expressing the DE1234 mutant annexin A4 (FIG. 2C).

When the bacteria were transformed with the plasmid expressing the DE1234 mutant (FIG. 2C), the cells formed longer filaments than were seen with the plasmid without an insert, suggesting the DE1234 quadruple mutant had a slight dominant negative effect on cytokinesis. However, these longer filaments tended to aggregate, which may have been due to partial lysis of the cells, and this may have prevented a precise statistical analysis of the filament lengths (FIG. 2C).

Bacterial cultures in ampicillin and piperacillin expressing the DE12 mutant annexin A4 (FIG. 2D).

When the bacteria were transformed with the plasmid expressing the DE12 mutant in medium containing ampicillin plus piperacillin, the filaments were significantly longer than normal, typically 10 to 36 μm (average 18.17 μm, SD 8.633 μm, n=60) (FIG. 2D). Therefore, the loss of the first two calcium binding sites apparently significantly impaired the ability of the annexin to promote cytokinesis in the presence of piperacillin.

Bacterial cultures in ampicillin and piperacillin expressing the DE34 mutant annexin A4 (FIG. 2E).

When the bacteria were transformed with the expression vector for the DE34 mutant the predominant length of the filaments was reduced to 5 to 10 μm (average 5.941 μm, SD 2.241 μm, n=72) in piperacillin plus ampicillin, indicating, as before with ampicillin alone, the mutants with intact first and second calcium binding domains alone were more effective at promoting cytokinesis than mutants that had only the intact third and fourth calcium binding domains.

Bacterial cultures in ampicillin and piperacillin expressing the DE234 mutant annexin A4 (FIG. 2F).

When the bacteria were transformed with the plasmid expressing the DE234 mutant, the cells formed predominantly short filaments of 2 to 8 μm length (average 4.601 μm, SD 1.723 μm, n=64) in ampicillin plus piperacillin, indicating once again, that the presence of only a single functional calcium binding site in domain 1 was sufficient to promote cytokinesis comparable to, or slightly greater than the extent that was promoted by the wild type annexin.

Statistical summary of filament lengths of cells grown in ampicillin (200 μg/ml) plus piperacillin (4 μg/ml.

Summary statistics for filaments grown in ampicillin and piperacillin are given in Table 3. P values for the differences between filament lengths of cells expressing different annexin constructs are given in Table 4. The behavior of the constructs fell into two distinct groups: Constructs that promoted cytokinesis (wild type annexin, DE34, and DE234) and constructs that did not promote cytokinesis (Ø (the null vector), DE12, and DE1234). The first group all expressed an annexin with a native (unmutated) first domain calcium binding site. The second group all had a mutated or absent first domain calcium binding site. The P values for comparisons of members of the first group with members of the second group were all highly significant: P values of 0.005 or less.

TABLE 3
Filament lengths in FIG. 2 (ampicillin plus piperacillin).

| Micrograph | Construct | n | AVG μm | SD μm | MAX μm |
|---|---|---|---|---|---|
| FIG. 2A | Ø | 43 | 10.71 | 5.520 | 29.79 |
| FIG. 2B | wt | 81 | 5.052 | 1.594 | 9.472 |
| FIG. 2C | DE1234 | — | — | — | — |
| FIG. 2D | DE12 | 60 | 18.17 | 8.633 | 36.28 |
| FIG. 2E | DE34 | 72 | 5.941 | 2.241 | 13.25 |
| FIG. 2F | DE234 | 64 | 4.601 | 1.723 | 8.718 |

Micrograph: Individual images in FIG. 2. Construct: Annexin expression construct: Ø, null vector with no annexin insert; wt, wildtype annexin A4; DE1234, etc -specific DE mutants with the mutated domains (1 through 4) indicated. n: Number of filaments measured in the designated micrograph. AVG μm: average length of the measured filaments; SD μm: sample standard deviation of the lengths of filaments; MAX μm: length of the longest filament in the micrograph. Note: Measurements were not made of filaments in FIG. 2C because of aggregation of the filaments (see text).

TABLE 4
Statistical analysis of the differences in filament
lengths in Table 3 (ampicillin plus piperacillin).

| | | | |
|---|---|---|---|
| Ø − wt | | | |
| 10.71, 5.052 | | | |
| P < 0.0001 | | | |
| Ø − DE12 | wt − DE12 | | |
| 10.71, 18.17 | 5.052, 18.17 | | |
| P < 0.0001 | P < 0.0001 | | |
| Ø − DE34 | wt − DE34 | DE12 − DE34 | |
| 10.71, 5.941 | 5.052, 5.941 | 18.17, 5.941 | |
| P < 0.0001 | P = 0.0050 | P < 0.0001 | |
| Ø − DE234 | wt − DE234 | DE12 − DE234 | DE34 − DE234 |
| 10.71, 4.601 | 5.052, 4.601 | 18.17, 4.601 | 5.941, 4.601 |
| P < 0.0001 | P = 0.1048 | P < 0.0001 | P = 0.0002 |

Each entry in the table is organized as is the data in the top left entry: Ø − wt compares the lengths of filaments harboring the null vector (Ø, no annexin) and filaments expressing the wild-type annexin (wt); 10.71, 5.052 compares the lengths of the two types of filaments; P < 0.0001, P value for the significance of the difference in lengths. Calculations made from data in Table 3.

Studies with ampicillin (200 μg/ml) plus cephalexin (25 μg/ml) (FIG. 3).

The antibiotic cephalexin has one of the highest affinities among the beta-lactam antibiotics for binding to penicillin binding protein 3 and was found in this study to have the greatest propensity to promote the formation of long, stable filaments, compared with ampicillin and piperacillin. When bacteria transformed with the empty expression plasmid (FIG. 3A), or with the wild type annexin (FIG. 3B), or the DE1234 mutant (FIG. 3D) were grown in ampicillin supplemented with cephalexin, long filamentous cells were formed over time, suggesting cytokinesis was blocked nearly completely. Many of the filaments extended beyond the field of view in the micrographs and the lengths were not measured. However, the filaments formed with the DE234 mutant (FIG. 3C) were of limited length at the same time point (3 hours and 20 minutes after seeding the cultures) and were estimated to be 15 to at least 100 μm in length. These filaments were observed over time to be growing more slowly than the other constructs, apparently because both growth and cytokinesis were occurring simultaneously.

Although the length of the filaments of the DE234 mutant cells grew more slowly than the lengths of the other constructs, in all cases the optical densities of the cultures increased at approximately the same rate, plus or minus approximately 20%. Apparently, the mass of total bacterial cells in the culture was increasing at a similar rate independent of the presence of the mutations, but the mass was differentially distributed between cells in the extended, filamentous form and the more normal shape as shorter rods, without having a large effect on the relationship between optical density and mass of cell material present.

Tenfold dilution of the cephalexin concentration results in an increase in cytokinesis in the filaments formed with the DE234 mutant, but not in the filaments formed with other mutants (FIG. 3E and 3F).

After 3 hours and 20 minutes of growth in the presence of ampicillin and cephalexin the cultures were diluted tenfold in fresh medium containing ampicillin but no cephalexin, resulting in a cephalexin concentration reduced from 25 to 2.5 μg/ml to test the stability of the filaments. The filaments formed by bacterial cultures harboring the empty plasmid, wild type annexin, or the DE1234 quadruple mutant were completely stable for the next two hours, unaffected by the reduction in cephalexin concentration (not shown). However, the cells expressing the DE234 mutant, containing only one functional calcium binding site, began to undergo cytokinesis to form shorter filaments after one hour, and further divided by two hours after the dilution. (Compare FIG. 3C with 3E and 3F). This activity of the DE234 mutant parallels the superior ability of the same mutant to promote cytokinesis in ampicillin alone or in ampicillin plus piperacillin seen in FIGS. 1 and 2 above.

### Electron Microscopy of Cells Expressing Wild Type or Mutant Annexins A4

Cultures of the BL21(DE3) cells harboring the empty expression plasmid or plasmids expressing wild-type or selected mutant annexins A4 were grown in LB medium plus 200 μg/ml ampicillin and 4 μg/ml piperacillin and processed for electron microscopy of ultrathin sections of pelleted cells to determine if the expression of the annexin altered the ultrastructure of the cells, particularly at sites of cell division. No unique morphology was apparent at sites of cell division with or without annexin expression. There was no apparent increase of protein density at division sites as might be expected if the annexin were preferentially accumulated at sites of cell division and membrane fusion. However, in all of these experiments the expression level of the annexin was necessarily kept low since high levels of annexin expression resulted in disruption of membrane structure and aggregation of the bacteria, as described in the Methods section.

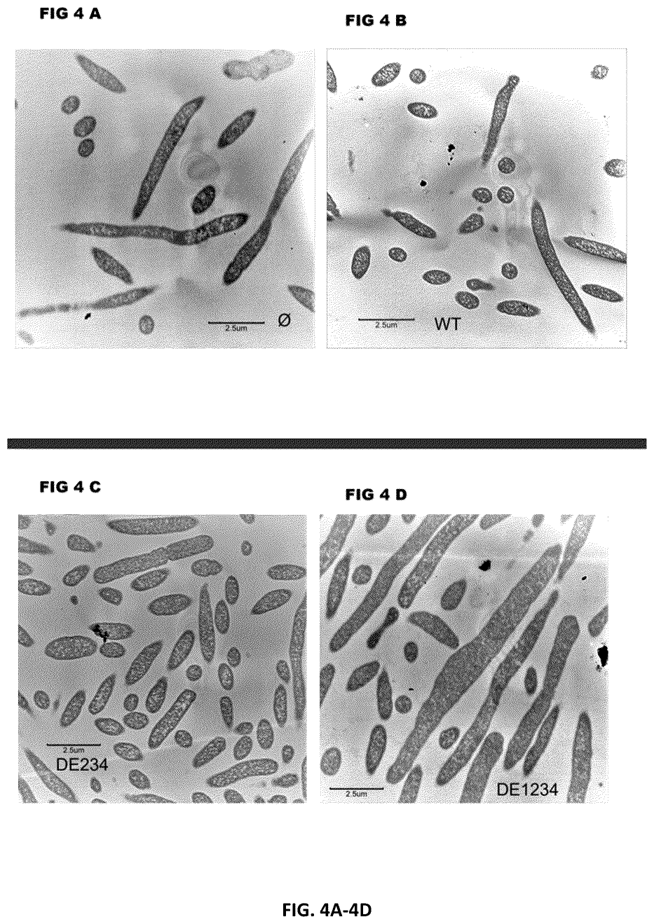

Although no unique ultrastructure was seen in cells expressing the annexin, the changes in filament length due to the annexin, as seen by light microscopy, were confirmed in the electron micrographs. FIG. 4A shows the morphology of cells harboring the empty expression plasmid and therefore without any annexin. This representative figure shows that there are both short bacterial cells present and filamentous forms, as was seen in the light micrographs of these cultures. FIG. 4B shows cells expressing the wild type annexin A4. In this case the number of short cells exceeds the number of extended filamentous forms due to the cytokinesis enhancing activity of the annexin. Similarly, in FIG. 4C the number of short cells exceeds the filamentous forms due to the cytokinesis promoting activity of the DE234 mutant, while in FIG. 4D the filamentous forms exceed the short forms due to the enhancement of filamentous forms due to the action of the DE1234 mutant. In these electron micrographs of ultrathin sections, it is not possible to determine if what appear to be short cells in the electron micrographs are not actually cross sections of the filamentous forms. However, light micrographs of similar cultures (e.g., in FIG. 2) show the complete cells and support the relative abundance of short and long forms in the cultures as interpreted in the electron micrographs.

### Discussion

Provided herein is the unexpected finding that a eukaryotic calcium-and membrane-binding protein, annexin A4, can enhance cytokinesis in bacteria in which cytokinesis is partially inhibited by beta-lactam antibiotics. The processes of cytokinesis in eukaryotes and in prokaryotes have in common characteristic morphological steps. The dividing cells pinch down into a neck between the forming daughter cells, the membranes in the neck fuse together and the cells separate. The molecular events underlying these morphological changes are under intense study in both cell types [9,23-25]. Different, unrelated sets of proteins undergo complex interactions in each of these types of organisms to mediate cytokinesis, but there is little similarity or known homology between the sets of proteins. In prokaryotes like E. coli a master protein appears to be FtsZ which has some features in common with the eukaryotic protein tubulin in that it has a GTPase activity and undergoes polymerization [25]. The FtsZ polymer forms a ring around the middle of the bacterium that contracts to form the neck between the dividing cells leading to membrane fusion and separation of the cells. However, to accomplish this the protein interacts with numerous other proteins, some of which, like penicillin binding protein 3 (PB3), bind beta-lactam antibiotics. The set of proteins responsible for cytokinesis in eukaryotes is also complex and the complete sequence of protein interactions is not fully worked out. Beta-lactam antibiotics do not interact with the proteins participating in cytokinesis in eukaryotic cells, indicating a significant contrast between the mechanisms in the two types of cells. These differences raise the question of how a single protein, annexin A4, can influence cytokinesis processes involving such distinct protein machineries.

The answer may be that the annexin interacts with two critical elements common to both types of cells: intracellular calcium and phospholipids [6]. The importance of the interaction with calcium in bacteria is demonstrated in this report since disabling the calcium binding sites in the annexin reduces its ability to promote cytokinesis. The interaction of the annexin with calcium is intimately linked to its interactions with phospholipids since both of these ligands participate in forming the high affinity calcium and phospholipid binding site in the annexin [8]. Since the binding of calcium and phospholipid has been demonstrated in the simplest model systems, involving only an annexin, calcium, and lipid bilayers, to lead to bilayer fusion [26], no other proteins may be necessary to support membrane fusion in cells. However, evidently other proteins are important in both eukaryotes and prokaryotes for membrane fusion to be utilized in the biological context of cytokinesis.

On the other hand, annexins are not absolutely required for cytokinesis in prokaryotes as demonstrated by the absence of annexins in the majority of bacteria [6]. A few eukaryotes, such as the yeast S. cerevisiae, can also carry out cytokinesis without depending on annexin homologues since they are not expressed in this yeast. Other mechanisms must be able to substitute for the role of the annexins in these counterexamples.

Although the exact mechanism by which the annexin promotes cytokinesis in bacteria is not clear, this does not prevent consideration of biotechnology applications of bacterial expression of annexins. As discussed in the Introduction, disassembly of bacterial filaments formed in the presence of antibiotics can promote the effectiveness of antibiotic chemotherapy by enabling cells of the immune system to destroy the non-filamentous forms of the bacteria [1,16]. Among the proteins studied here, the DE234 mutant annexin A4 might be the most effective therapeutic because of its efficacy in promoting cytokinesis in the bacterial filaments.

The ability of annexins to disrupt the formation of filaments by bacteria might also have application in the prevention of “biofilms”. These are colonies of bacteria that form on surfaces and are typically encased in a polysaccharide matrix [28]. They are ubiquitous in nature and can influence disease progression in a number of pathologies such as cystic fibrosis, native valve endocarditis, otitis media, periodontitis, and chronic prostatitis. The formation of biofilms on Implanted medical devices can result in device failure [29]. The initial formation of biofilms is thought to frequently be caused by the formation of bacterial filaments which then assemble to form the biofilm [30]. The same antibiotics that lead to filament growth in patients are associated with the development of biofilms, supporting the concept that filament formation may be a precursor of biofilm formation. Therefore, inhibition of filament formation or stability by the introduction of annexins may provide a method of treating biofilm associated pathologies.

The possibility of using annexins to suppress the deleterious effects of pathogenic bacteria will be dependent on the ability to express the eukaryotic annexins in the bacteria while they are invading a host. Multiple approaches may be available, including transfection by expression plasmids as used in this study, or introduction through phage-based transfer of genetic material [31].

### Conclusions

A eukaryotic calcium-and membrane-binding protein, bovine annexin A4, is shown here to promote cytokinesis in bacterial cells in which this process has been partially inhibited by beta-lactam antibiotics. The expression of annexins in bacteria may therefore be a useful adjuvant in bacterial chemotherapy as it might make the shortened bacterial cells more susceptible to attack by cells of the immune system. The similar calcium-and membrane-binding properties of other annexins suggest that promotion of cytokinesis might be a common property of all annexins.

### BIBLIOGRAPHY

[1] S.S. Justice, D.A. Hunstad, L. Cegelski, S.J. Hultgren, Morphological plasticity as a bacterial survival strategy., Nat. Rev. Microbiol. 6 (2008) 162-168. https://doi.org/10.1038/nrmicro1820.
[2] C.E. Creutz, C.J. Pazoles, H.B. Pollard, Identification and purification of an adrenal medullary protein (synexin) that causes calcium-dependent aggregation of isolated chromaffin granules., J. Biol. Chem. 253 (1978) 2858-2866.
[3] V. Gerke, C.E. Creutz, S.E. Moss, Annexins: linking Ca2+ signalling to membrane dynamics., Nat. Rev. Mol. Cell Biol. 6 (2005) 449-461. https://doi.org/10.1038/nrm1661.
[4] M.J. Geisow, U. Fritsche, J.M. Hexham, B. Dash, T. Johnson, A consensus amino-acid sequence repeat in Torpedo and mammalian Ca2+-dependent membrane-binding proteins., Nature. 320 (1986) 636-638. https://doi.org/10.1038/320636a0.
[5] R.O. Morgan, S. Martin-Almedina, M. Garcia, J. Jhoncon-Kooyip, M .-P. Fernandez, Deciphering function and mechanism of calcium-binding proteins from their evolutionary imprints., Biochim. Biophys. Acta. 1763 (2006) 1238-1249. https://doi.org/10.1016/j.bbamcr.2006.09.028.
[6] P.K. Kodavali, M. Dudkiewicz, S. Pikuła, K. Pawłowski, Bioinformatics analysis of bacterial annexins—putative ancestral relatives of eukaryotic annexins., PLOS ONE. 9 (2014) e85428. https://doi.org/10.1371/journal.pone.0085428.
[7] M. Junker, C.E. Creutz, Ca(2+)-dependent binding of endonexin (annexin IV) to membranes: analysis of the effects of membrane lipid composition and development of a predictive model for the binding interaction., Biochemistry. 33 (1994) 8930-8940. https://doi.org/10.1021/bi00196a010.
[8] M.A. Swairjo, N.O. Concha, M.A. Kaetzel, J.R. Dedman, B.A. Seaton, Ca(2+)-bridging mechanism and phospholipid head group recognition in the membrane-binding protein annexin V., Nat. Struct. Biol. 2 (1995) 968-974. https://doi.org/10.1038/nsb1195-968.
[9] A. Tomas, C. Futter, S.E. Moss, Annexin 11 is required for midbody formation and completion of the terminal phase of cytokinesis., J. Cell Biol. 165 (2004) 813-822. https://doi.org/10.1083/jcb.200311054.
[10] C.E. Creutz, cis-Unsaturated fatty acids induce the fusion of chromaffin granules aggregated by synexin., J. Cell Biol. 91 (1981) 247-256. https://doi.org/10.1083/jcb.91.1.247.
[11] G.D. Gardner, Morphological Effects of Penicillin on Bacteria, Nature. 146 (1940) 837-838.
[12] E. Chain, H.W. Florey, A.D. Gardner, N.G. Heatley, M.A. Jennings, J. Orr-Ewing, A.G. Sanders, Penicillin as a chemotherapeutic agent, Lancet. (1940) 226-228.
[13] J. Buijs, A.S.M. Dofferhoff, J.W. Mouton, J.H.T. Wagenvoort, J.W.M. van der Meer, Concentration-dependency of beta-lactam-induced filament formation in Gram-negative bacteria., Clin. Microbiol. Infect. 14 (2008) 344-349. https://doi.org/10.1111/j.1469-0691.2007.01940.x.
[14] D.J. Waxman, J.L. Strominger, Penicillin-binding proteins and the mechanism of action of beta-lactam antibiotics., Annu. Rev. Biochem. 52 (1983) 825-869. https://doi.org/10.1146/annurev.bi.52.070183.004141.
[15] B.G. Spratt, Distinct penicillin binding proteins involved in the division, elongation, and shape of Escherichia coli K12., Proc Natl Acad Sci USA. 72 (1975) 2999-3003. https://doi.org/10.1073/pnas.72.8.2999.
[16] S.S. Justice, D.A. Hunstad, P.C. Seed, S.J. Hultgren, Filamentation by Escherichia coli subverts innate defenses during urinary tract infection., Proc Natl Acad Sci USA. 103 (2006) 19884-19889. https://doi.org/10.1073/pnas.0606329104.
[17] M.R. Nelson, C.E. Creutz, Combinatorial mutagenesis of the four domains of annexin IV: effects on chromaffin granule binding and aggregating activities., Biochemistry. 34 (1995) 3121-3132. https://doi.org/10.1021/bi00009a044.
[18] M.R. Nelson, Structure-function analysis of recombinant annexin IV using site-directed mutagenesis. Ph.D. Thesis, University of Virginia, Doctoral dissertation, University of Virginia, 1993.
[19] F.W. Studier, A.H. Rosenberg, J.J. Dunn, J.W. Dubendorff, Use of T7 RNA polymerase to direct expression of cloned genes., Meth. Enzymol. 185 (1990) 60-89.
[20] M.R. Nelson, C.E. Creutz, Comparison of the expression of native and mutant bovine annexin IV in Escherichia coli using four different expression systems., Protein Expr. Purif. 6 (1995) 132-140. https://doi.org/10.1006/prep.1995.1016.
[21] G.A. Botta, J.T. Park, Evidence for Involvement of Penicillin-Binding Protein 3 inMurein Synthesis During Septation but Not During Cell elongation, J Bacteriol. 145 (1981) 333-340.
[22] C.E. Creutz, S.L. Snyder, S.N. Daigle, J. Redick, Identification, localization, and functional implications of an abundant nematode annexin., J. Cell Biol. 132 (1996) 1079-1092. https://doi.org/10.1083/jcb. 132.6.1079.
[23] L.I. Rothfield, S.S. Justice, Bacterial cell division: the cycle of the ring., Cell. 88 (1997) 581-584. https://doi.org/10.1016/s0092-8674(00)81899-1.
[24] J. Errington, R.A. Daniel, D.-J. Scheffers, Cytokinesis in bacteria., Microbiol. Mol. Biol. Rev. 67 (2003) 52-65, table of contents. https://doi.org/10.1128/MMBR.67.1.52-65.2003.
[25] C. Deschamps, A. Echard, F. Niedergang, Phagocytosis and cytokinesis: do cells use common tools to cut and to eat? Highlights on common themes and differences., Traffic. 14 (2013) 355-364. https://doi.org/10.1111/tra.12045.
[26] C. Coltharp, J. Buss, T.M. Plumer, J. Xiao, Defining the rate-limiting processes of bacterial cytokinesis., Proc Natl Acad Sci USA. 113 (2016) E1044-53. https://doi.org/10.1073/pnas.1514296113.
[27] K. Hong, N. Düzgüneş, R. Ekerdt, D. Papahadjopoulos, Synexin facilitates fusion of specific phospholipid membranes at divalent cation concentrations found intracellularly., Proc Natl Acad Sci USA. 79 (1982) 4642-4644. https://doi.org/10.1073/pnas.79.15.4642.
[28] R.M. Donlan, Biofilms: microbial life on surfaces., Emerging Infect. Dis. 8 (2002) 881-890. https://doi.org/10.3201/eid0809.020063.
[29] R.M. Donlan, Biofilms and device-associated infections., Emerging Infect. Dis. 7 (2001) 277-281. https://doi.org/10.3201/eid0702.010226.
[30] Y. Jin, H. Zheng, A.C.S. Ibanez, P.D. Patil, S. Lv, M. Luo, T.M. Duncan, Y.-Y. Luk, Cell-Wall-Targeting Antibiotics Cause Lag-Phase Bacteria to Form Surface-Mediated Filaments Promoting the Formation of Biofilms and Aggregates., Chembiochem. 21 (2020) 825-835. https://doi.org/10.1002/cbic.201900508.
[31] K.E. Kortright, B.K. Chan, J.L. Koff, P.E. Turner, Phage Therapy: A Renewed Approach to Combat Antibiotic-Resistant Bacteria., Cell Host Microbe. 25 (2019) 219-232. https://doi.org/10.1016/j.chom.2019.01.014.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A composition consisting essentially of an annexin polypeptide or subunit thereof and an antibiotic.

2. A method to breakdown bacterial filaments in bacteria comprising contacting or expressing an annexin or subunit thereof in said bacteria so that the bacterial filaments are shorter than those of a bacterium that are not contacted with or expressing said annexin or subunit thereof.

3. The method of claim 2, wherein the bacteria are exposed to an external stress.

4. The method of claim 3, wherein the external stress is exposure to an antibiotic, malnutrition, chemical, osmolarity, pH, temperature or combination thereof.

5. The method of claim 4, wherein the stress comprises an antibiotic.

6. The method of claim 2, wherein the antibiotic comprises a beta lactam.

7. The method of claims 2, wherein the bacteria are pathogenic bacteria.

8. The method of claim 2, wherein the annexin comprises an annexin A4.

9. The method of claim 2, wherein the subunit comprises a single annexin repeat.

10. A method to increase phagocytosis of bacteria that have been exposed to an external stress comprising contacting or expressing an annexin or subunit thereof in said bacteria thereby rendering the bacteria more susceptible to phagocytosis as compared to bacteria that have been exposed to an external stress and are not contacted with or expressing said annexin or subunit thereof.

11. The method of claim 10, wherein the external stress is exposure to an antibiotic, malnutrition, chemical, osmolarity, pH, temperature or combination thereof.

12. The method of claim 11, wherein the stress comprises an antibiotic.

13. A method to increase the efficacy of an antibiotic comprising contacting or expressing an annexin polypeptide or subunit thereof in bacteria wherein said bacteria are also exposed to said antibiotic, wherein the efficacy of the antibiotic is increased as compared to bacteria not expressing or contacted with said annexin.

14. The method of claim 13, wherein the contacting comprises administering said annexin polypeptide or subunit thereof and an antibiotic to a subject in need thereof.

15. The method of claim 14, wherein the annexin polypeptide or subunit thereof is administered in a vector.

16. The method of claim 13, wherein the bacteria are pathogenic.

17. The method of claim 13, wherein the antibiotic is a beta lactam.

18. A method to inhibit or reduce the development of antibiotic-resistant bacteria comprising contacting or expressing an annexin polypeptide or a subunit thereof in said bacteria wherein said bacteria are also exposed to said antibiotic, wherein the bacteria are inhibited as compared to bacteria not expressing or contacted with said annexin.

19. The method of claim 18, wherein the contacting comprises administering said annexin polypeptide or subunit thereof and an antibiotic to a subject in need thereof.

20. The method of claim 19, wherein the annexin polypeptide or subunit thereof is administered in a vector.
